(19) United States
(12) Patent Application Publication
(10) Pub. No.: US 2005/0059149 A1
(43) Pub. Date: Mar. 17, 2005

(54) Methods of ex-vivo expansion of hematopoeitic cells using multivariant IL-3 hematopoiesis chimera proteins

(76) Inventors: S. Christopher Bauer, New Haven, MO (US); Mark Allen Abrams, St. Louis, MO (US); Sarah Ruth Braford-Goldberg, St. Louis, MO (US); Maire Helena Caparon, Chesterfield, MO (US); Alan Michael Easton, Maryland Heights, MO (US); Barbara Kure Klein, St. Louis, MO (US); John P. McKearn, Glencoe, MO (US); Peter O. Olins, Lafayette, CO (US); Kumnan Paik, Wilmette, IL (US); John W. Thomas, Town & Country, MO (US)

Correspondence Address: Winstead Sechrest & Minick P.C., P.O. Box 50784, Dallas, TX, 75201, US

(21) Appl. No.: 10/948,576

(22) Filed: Sep. 22, 2004

Related U.S. Application Data
(63) Continuation of application No. 10/083,446, filed on Feb. 26, 2002, now abandoned.
Application No. 10/083,446 is a division of application No. 08/762,227, filed on Dec. 9, 1996, now Pat. No. 6,436,387.
Application No. 08/762,227 is a continuation-in-part of application No. 08/446,872, filed on Jun. 6, 1995, now Pat. No. 6,361,977.
Application No. 08/446,872 is a continuation of application No. 08/192,325, filed on Feb. 4, 1994, now Pat. No. 6,057,133.

(30) Foreign Application Priority Data
Nov. 22, 1993 (WO) PCT/US93/11197

(51) Int. Cl. C12N 5/08

(52) U.S. Cl. 435/368; 435/404; 424/85.2

(57) ABSTRACT

The present invention relates to methods of ex-vivo expansion of hematopoietic cells by culturing hematopoietic cells in a growth medium comprising a chimera protein which comprises a variant of human interleukin-3 (hIL-3) which contains multiple amino acid substitutions and which may have portions of the native hIL-3 molecule deleted and a hematopoietic growth factor. The present invention also relates to the ex-vivo expansion of hematopoietic cells for gene therapy. Additionally, the present invention relates to the use of the expanded hematopoietic cells for treating patients having a hematopoietic disorder.

### CROSS REFERENCES TO RELATED APPLICATIONS

This is a continuation application of Ser. No. 10/083,446, which was filed Feb. 26, 2002, which is a divisional application of Ser. No. 08/762,227, which was filed Dec. 09, 1996, now U.S. Pat. No. 6,436,387, which is a continuation-in-part of Ser. No. 08/446,872, filed Jun. 06, 1995, now U.S. Pat. No. 6,361,977, which was filed under 35 U.S.C. § 371 from PCT/US95/01185 filed Feb. 02, 1995, which is a continuation-in-part of Ser. No. 08/192,325, filed Feb. 04, 1994, now U.S. Pat. No. 6,057,133, which is a continuation-in-part of Ser. No. 08/411,795, filed Apr. 06, 1995, now U.S. Pat. No. 5,604,116, which was filed under 35 U.S.C. § 371 from PCT/US93/11197, filed Nov. 22, 1993, which is a continuation-in-part of Ser. No. 07/981,044 filed Nov. 24, 1992, now abandoned. The noted applications are incorporated herein by reference.

### FIELD OF THE INVENTION

The present invention relates to methods of ex-vivo expansion of hematopoietic cells by culturing hematopoietic cells in a medium which includes a chimera protein comprising a variant of human interleukin-3 (hIL-3) joined with or without a linker to a second colony stimulating factors, cytokines, lymphokines, interleukins, hematopoietic growth factors or IL-3 variants and the use of the expanded hematopoietic cells for treating patients having a hematopoietic disorder.

### BACKGROUND OF THE INVENTION

Colony stimulating factors, cytokines, lymphokines, interleukins or hematopoietic growth factors (herein collectively referred to as “hematopoietic growth factors”) which stimulate the differentiation and/or proliferation of bone marrow cells have generated much interest because of their therapeutic potential for restoring depressed levels of hematopoietic stem cell-derived cells. Hematopoietic growth factors in both human and murine systems have been identified and distinguished according to their activities. For example, granulocyte-CSF (G-CSF) and macrophage-CSF (M-CSF) stimulate the in vitro formation of neutrophilic granulocyte and macrophage colonies, respectively while GM-CSF and interleukin-3 (IL-3) have broader activities and stimulate the formation of both macrophage, neutrophilic and eosinophilic granulocyte colonies. IL-3 also stimulates the formation of mast, megakaryocyte and pure and mixed erythroid colonies.

Because of its ability to stimulate the proliferation of a number of different cell types and to support the growth and proliferation of progenitor cells, IL-3 has potential for therapeutic use in restoring hematopoietic cells to normal amounts in those cases where the number of cells has been reduced due to diseases or to therapeutic treatments such as radiation and/or chemotherapy.

Interleukin-3 (IL-3) is a hematopoietic growth factor which has the property of being able to promote the survival, growth and differentiation of hematopoietic cells. Among the biological properties of IL-3 are the ability (a) to support the growth and differentiation of progenitor cells committed to all, or virtually all, blood cell lineages; (b) to interact with early multipotential stem cells; (c) to sustain the growth of pluripotent precursor cells; (d) to stimulate proliferation of chronic myelogenous leukemia (CML) cells; (e) to stimulate proliferation of mast cells, eosinophils and basophils; (f) to stimulate DNA synthesis by human acute myelogenous leukemia (AML) cells; (g) to prime cells for production of leukotrienes and histamines; (h) to induce leukocyte chemotaxis; and (i) to induce cell surface molecules needed for leukocyte adhesion.

Mature human interleukin-3 (hIL-3) consists of 133 amino acids. It has one disulfide bridge and two potential glycosylation sites (Yang, et al., CELL 47:3 (1986)).

Murine IL-3 (mIL-3) was first identified by Ihle, et al., J. IMMUNOL. 126:2184 (1981) as a factor which induced expression of a T cell associated enzyme, 20_-hydroxysteroid dehydrogenase. The factor was purified to homogeneity and shown to regulate the growth and differentiation of numerous subclasses of early hematopoietic and lymphoid progenitor cells.

In 1984, cDNA clones coding for murine IL-3 were isolated (Fung, et al., NATURE 307:233 (1984) and Yokota, et al., PROC. NATL. ACAD. SCI. USA 81:1070 (1984)). The murine DNA sequence coded for a polypeptide of 166 amino acids including a putative signal peptide.

The gibbon IL-3 sequence was obtained using a gibbon cDNA expression library. The gibbon IL-3 sequence was then used as a probe against a human genomic library to obtain a human IL-3 sequence.

Gibbon and human genomic DNA homologues of the murine IL-3 sequence were disclosed by Yang, et al., CELL 47:3 (1986). The human sequence reported by Yang, et al. included a serine residue at position 8 of the mature protein sequence. Following this finding, others reported isolation of Pro⁸ hIL-3 cDNAs having proline at position 8 of the protein sequence. Thus it appears that there may be two allelic forms of hIL-3.

Dorssers, et al., GENE 55:115 (1987), found a clone from a human cDNA library which hybridized with mIL-3. This hybridization was the result of the high degree of homology between the 3′ noncoding regions of mIL-3 and hIL-3. This cDNA coded for an hIL-3 (Pro⁸) sequence.

U.S. Pat. No. 4,877,729 and U.S. Pat. No. 4,959,454 disclose human IL-3 and gibbon IL-3 cDNAs and the protein sequences for which they code. The hIL-3 disclosed has serine rather than proline at position 8 in the protein sequence.

Clark-Lewis, et al., SCIENCE 231:134 (1986) performed a functional analysis of murine IL-3 analogs synthesized with an automated peptide synthesizer. The authors concluded that the stable tertiary structure of the complete molecule was required for full activity. A study on the role of the disulfide bridges showed that replacement of all four cysteines by alanine gave a molecule with 1/500th the activity as the native molecule. Replacement of two of the four Cys residues by Ala(Cys⁷⁹, Cys¹⁴⁰->Ala⁷⁹, Ala¹⁴⁰) resulted in an increased activity. The authors concluded that in murine IL-3 a single disulfide bridge is required between cysteines 17 and 80 to get biological activity that approximates physiological levels and that this structure probably stabilizes the tertiary structure of the protein to give a conformation that is optimal for function. (Clark-Lewis, et al., PROC. NATL. ACAD. SCI. USA 85:7897 (1988)).

International Patent Application (PCT) WO 88/00598 discloses gibbon- and human-like IL-3. The hIL-3 contains a Ser⁸->Pro⁸ replacement. Suggestions are made to replace Cys by Ser, thereby breaking the disulfide bridge, and to replace one or more amino acids at the glycosylation sites.

EP-A-0275598 (WO 88/04691) illustrates that Ala¹ can be deleted while retaining biological activity. Some mutant hIL-3 sequences are provided, e.g., two double mutants, Ala¹->Asp¹, Trp¹³->Arg¹³ (pGB/IL-302) and Ala¹->Asp¹, Met³->Thr³ (pGB/IL-304) and one triple mutant Ala¹->Asp¹, Leu⁹->Pro⁹, Trp¹³->Arg¹³ (pGB/IL-303).

WO 88/05469 describes how deglycosylation mutants can be obtained and suggests mutants of Arg⁵⁴Arg⁵⁵ and Arg¹⁰⁸Arg¹⁰⁹Lys¹¹⁰ might avoid proteolysis upon expression in Saccharomyces cerevisiae by KEX2 protease. No mutated proteins are disclosed. Glycosylation and the KEX2 protease activity are only important, in this context, upon expression in yeast.

WO 88/06161 mentions various mutants which theoretically may be conformationally and antigenically neutral. The only actually performed mutations are Met²->Ile² and Ile¹³¹->Leu¹³¹. It is not disclosed whether the contemplated neutralities were obtained for these two mutations.

WO 91/00350 discloses nonglycosylated hIL-3 analog proteins, for example, hIL-3 (Pro⁸Asp¹⁵Asp⁷⁰), Met³ rhuIL-3 (Pro⁸Asp¹⁵Asp⁷⁰); Thr⁴ rhuIL-3 (Pro⁸Asp¹⁵Asp⁷⁰)and Thr⁶ rhuIL-3 (Pro⁸Asp¹⁵Asp⁷⁰). It is said that these protein compositions do not exhibit certain adverse side effects associated with native hIL-3 such as urticaria resulting from infiltration of mast cells and lymphocytes into the dermis. The disclosed analog hIL-3 proteins may have N termini at Met³, Thr⁴, or Thr⁶.

WO 91/12874 discloses cysteine added variants (CAVs) of IL-3 which have at least one Cys residue substituted for a naturally occurring amino acid residue.

U.S. Pat. No. 4,810,643 discloses the DNA sequence encoding human G-CSF.

WO 91/02754 discloses a fusion protein composed of GM-CSF and IL-3 which has increased biological activity compared to GM-CSF or IL-3 alone. Also disclosed are nonglycosylated IL-3 and GM-CSF analog proteins as components of the fusion.

WO 92/04455 discloses fusion proteins composed of IL-3 fused to a lymphokine selected from the group consisting of IL-3, IL-6, IL-7, IL-9, IL-11, EPO and G-CSF.

WO 92/06006 relates to hematopoietic molecules comprising an early acting factor (IL-3 or GM-CSF) and a late acting factor (EPO, IL-5, G-CSF or M-CSF) and the in vivo use for treating hematopoietic disorders.

Hematopoietic growth factors, such as hIL-3, have been administered alone, co-administered with other hematopoietic growth factors, or in combination with bone marrow transplants subsequent to high dose chemotherapy to treat the neutropenia and thrombocytopenia which are often the result of such treatment. However the period of severe neutropenia and thrombocytopenia may not be totally eliminated. The myeloid lineage, which is comprised of monocytes (macrophages), granulocytes (including neutrophils) and megakaryocytes, is critical in preventing infections and bleeding which can be life-threatening. Neutropenia and thrombocytopenia may also be the result of disease, genetic disorders, drugs, toxins, radiation and many therapeutic treatments such as conventional oncology therapy.

Bone marrow transplants have been used to treat this patient population. However, several problems are associated with the use of bone marrow to reconstitute a compromised hematopoietic system including: 1) the number of stem cells in bone marrow or other is limited, 2) Graft Versus Host Disease, 3) graft rejection and 4) possible contamination with tumor cells. Stem cells make up a very small percentage of the nucleated cells in the bone marrow, spleen and peripheral blood. It is clear that a dose response exits such that a greater number of stem cells will enhance hematopoietic recovery. Therefore, the use of hematopoietic cells that have been expanded ex-vivo should enhance hematopoietic recovery and patient survival. Bone marrow from an allogeneic donor has been used to provide bone marrow for transplant. However, Graft Versus Host Disease and graft rejection limit bone marrow transplantation even in recipients with HLA-matched sibling donors. An alternative to allogenic bone marrow transplants is autologous bone marrow transplants. In autologous bone marrow transplants, some of the patient's own marrow is harvested prior to myeloablative therapy, e.g. high dose chemotherapy, and is transplanted back into the patient afterwards. Autologous transplants eliminate the risk of Graft Versus Host Disease and graft rejection. However, autologous bone marrow transplants still present problems in terms of the limited number of stems cells in the marrow and possible contamination with tumor cells.

The limited number of stem cells may be overcome by ex-vivo expansion of the stem cells. In addition, stem cells can be specifically isolated selected based on the presence of specific surface antigen such as CD34+ in order to decrease tumor cell contamination of the marrow graft.

The following patents contain further details on separating stem cells, CD34+ cells, culturing the cells with hematopoietic growth factors, the use of the cells for the treatment of patients with hematopoietic disorders and the use of hematopoietic factors for cell expansion and gene therapy.

U.S. Pat. No. 5,061,620 relates to compositions comprising human hematopoietic stem cells provided by separating the stem cells from dedicated cells.

U.S. Pat. No. 5,199,942 describes a method for autologous hematopoietic cell transplantation comprising: (1) obtaining hematopoietic progenitor cells from a patient; (2) ex-vivo expansion of cells with a growth factor selected from the group consisting of IL-3, flt3 ligand, c-kit ligand, GM-CSF, IL-1, GM-CSF/IL-3 fusion protein and combinations thereof; (3) administering cellular preparation to a patient.

U.S. Pat. No. 5,240,856 relates to a cell separator that includes apparatus for automatically controlling the cell separation process.

U.S. Pat. No. 5,409,813 describes methods of positive and negative selection of a cell population from a mixture of cell populations utilizing a magnetically stabilized fluidized bed.

U.S. Pat. No. 5,409,825 relates to a method of growing hematopoietic stem cells in a liquid culture medium using mast cell growth factor (MGF) and optionally at least one cytokine selected from the group consisting of IL-3, GM-CSF and IL-3/GM-CSF fusion protein.

U.S. Pat. No. 5,459,069 relates to devices for maintaining and growing human stem cells and/or hematopoietic cells in culture.

U.S. Pat. No. 5,541,103 describes peripheral blood progenitor cells obtained by enriching blood progenitors expressing the cd34 antigen and culture the cells in a growth medium consisting of IL-1, IL-3, IL-6, erythropoietin and stem cell growth factor.

U.S. Pat. No. 5,464,753 describes a method of purifying pluripotent hematopoietic stem cells expressing P-glycoprotein from a mixture of blood or bone marrow cells.

U.S. Pat. No. 5, 547,687 relates to a method of enriching CD34 cells from a cell mixture by density centrifugation.

U.S. Pat. No. 5,571,686 depicts the use of megapoietin (c-mpl ligand) for the in vitro expansion of stem cells as a source of platelets for transplantation and for increasing the storage life of platelets.

WO 91/16116 describes devices and methods for selectively isolating and separating target cells from a mixture of cells.

WO 91/18972 describes methods for in vitro culturing of bone marrow, by incubating suspension of bone marrow cells, using a hollow fiber bioreactor.

WO 92/18615 relates to a process for maintaining and expanding bone marrow cells, in a culture medium containing specific mixtures of cytokines, for use in transplants.

WO 93/08268 describes a method for selectively expanding stem cells, comprising the steps of (a) separating CD34+ stem cells from other cells and (b) incubating the separated cells in a selective medium, such that the stem cells are selectively expanded.

WO 93/18136 describes a process for in vitro support of mammalian cells derived from peripheral blood.

WO 93/18648 relates to a composition comprising human neutrophil precursor cells with a high content of myeloblasts and promyelocytes for treating genetic or acquired neutropenia.

WO 94/08039 describes a method of enrichment for human hematopoietic stem cells by selection for cells which express c-kit protein.

WO 94/11493 describes a stem cell population that are CD34+ and small in size, which are isolated using a counterflow elutriation method.

WO 94/27698 relates to a method combining immunoaffinity separation and continuous flow centrifugal separation for the selective separation of a nucleated heterogeneous cell population from a heterogeneous cell mixture.

WO 94/25848 describes a cell separation apparatus for collection and manipulation of target cells.

The long term culturing of highly enriched CD34+ precursors of hematopoietic progenitor cells from human bone marrow in cultures containing IL-1α, IL-3, IL-6 or GM-CSF is discussed in Brandt et al., J. Clin. Invest. 86:932-941, 1990.

### SUMMARY OF THE INVENTION

The present invention encompasses the use of chimera proteins, comprising a recombinant human interleukin-3 (hIL-3) variant or mutant proteins (muteins) joined with or without a linker to a second colony stimulating factor (CSF), cytokine, lymphokine, interleukin, hematopoietic growth factor (herein collectively referred to as “hematopoietic growth factors”) or IL-3 variant, for the ex-vivo expansion of hematopoietic cells. These hIL-3 muteins contain amino acid substitutions and may also have amino acid deletions at either/or both the N- and C- termini. This invention encompasses mixed function hematopoietic growth factors formed from covalently linked polypeptides, each of which may act through a different and specific cell receptor to initiate complementary biological activities.

Novel compounds of this invention are represented by the formulas
R₁-L-R₂, R₂-L-R₁, R₁-R₂, R₂-R₁, R₁-L-R₁ and R₁-R₁
where R1 is a hIL-3 variant which contains multiple amino acid substitutions and which may have portions of the hIL-3 molecule deleted, R2 is an IL-3, IL-3 variant or hematopoietic growth factor with a different but complementary activity. The R1 polypeptide is joined either directly or through a linker segment to the R2 polypeptide. Thus L represents a chemical bond or polypeptide segment to which both R1 and R2 are joined. Preferably, these mutant IL-3 polypeptides of the present invention contain four or more amino acids which differ from the amino acids found at the corresponding positions in the native hIL-3 polypeptide.

These chimera molecules may be characterized by having the usual activity of both of the peptides forming the chimera molecule or it may be further characterized by having a biological or physiological activity greater than simply the additive function of the presence of IL-3 or the second hematopoietic growth factor alone. The chimera molecule may also unexpectedly provide an enhanced effect on the activity or an activity different from that expected by the presence of IL-3 or the second hematopoietic growth factor or IL-3 variant. The chimera molecule may also have an improved activity profile which may include reduction of undesirable biological activities associated with native hIL-3.

The present invention also includes mutants of hIL-3 in which from 1 to 14 amino acids have been deleted from the N-terminus and/or from 1 to 15 amino acids have been deleted from the C-terminus, containing multiple amino acid substitutions, to which a second hematopoietic growth factor or IL-3 variant has been joined. Preferred chimera molecules of the present invention are composed of hIL-3 variants in which amino acids 1 to 14 have been deleted from the N-terminus, amino acids 126 to 133 have been deleted from the C-terminus, and contains from about four to about twenty-six amino acid substitutions in the polypeptide sequence joined to second hematopoietic growth factor or IL-3 variant.

The present invention includes methods for selective ex vivo expansion of stem cells, comprising the steps of; (a) culturing said stem cells with a selected growth medium comprising a chimera protein having the formula selected from the group consisting of:
R₁-L-R₂, R₂-L-R₁, R₁-R₂, R₂-R₁, R₁-L-R₁ and R₁-R₁

wherein R₁ is a human interleukin-3 mutant polypeptide of SEQ ID NO:1

wherein

Xaa at position 17 is Ser, Lys, Gly, Asp, Met, Gln, or Arg;
Xaa at position 18 is Asn, His, Leu, Ile, Phe, Arg, or Gln;
Xaa at position 19 is Met, Phe, Ile, Arg, Gly, Ala, or Cys;
Xaa at position 20 is Ile, Cys, Gln, Glu, Arg, Pro, or Ala;
Xaa at position 21 is Asp, Phe, Lys, Arg, Ala, Gly, Glu, Gln, Asn, Thr, Ser or Val;
Xaa at position 22 is Glu, Trp, Pro, Ser, Ala, His, Asp, Asn, Gln, Leu, Val or Gly;
Xaa at position 23 is Ile, Val, Ala, Leu, Gly, Trp, Lys, Phe, Ser, or Arg;
Xaa at position 24 is Ile, Gly, Val, Arg, Ser, Phe, or Leu;
Xaa at position 25 is Thr, His, Gly, Gln, Arg, Pro, or Ala;
Xaa at position 26 is His, Thr, Phe, Gly, Arg, Ala, or Trp;
Xaa at position 27 is Leu, Gly, Arg, Thr, Ser, or Ala;
Xaa at position 28 is Lys, Arg, Leu, Gln, Gly, Pro, Val or Trp;
Xaa at position 29 is Gln, Asn, Leu, Pro, Arg, or Val;
Xaa at position 30 is Pro, His, Thr, Gly, Asp, Gln, Ser, Leu, or Lys;
Xaa at position 31 is Pro, Asp, Gly, Ala, Arg, Leu, or Gln;
Xaa at position 32 is Leu, Val, Arg, Gln, Asn, Gly, Ala, or Glu;
Xaa at position 33 is Pro, Leu, Gln, Ala, Thr, or Glu;
Xaa at position 34 is Leu, Val, Gly, Ser, Lys, Glu, Gln, Thr, Arg, Ala, Phe, Ile or Met;
Xaa at position 35 is Leu, Ala, Gly, Asn, Pro, Gln, or Val;
Xaa at position 36 is Asp, Leu, or Val;
Xaa at position 37 is Phe, Ser, Pro, Trp, or Ile;
Xaa at position 38 is Asn, or Ala;
Xaa at position 40 is Leu, Trp, or Arg;
Xaa at position 41 is Asn, Cys, Arg, Leu, His, Met, or Pro;
Xaa at position 42 is Gly, Asp, Ser, Cys, Asn, Lys, Thr, Leu, Val, Glu, Phe, Tyr, Ile, Met or Ala;
Xaa at position 43 is Glu, Asn, Tyr, Leu, Phe, Asp, Ala, Cys, Gln, Arg, Thr, Gly or Ser;
Xaa at position 44 is Asp, Ser, Leu, Arg, Lys, Thr, Met, Trp, Glu, Asn, Gln, Ala or Pro;
Xaa at position 45 is Gln, Pro, Phe, Val, Met, Leu, Thr, Lys, Trp, Asp, Asn, Arg, Ser, Ala, Ile, Glu or His;
Xaa at position 46 is Asp, Phe, Ser, Thr, Cys, Glu, Asn, Gln, Lys, His, Ala, Tyr, Ile, Val or Gly;
Xaa at position 47 is Ile, Gly, Val, Ser, Arg, Pro, or His;
Xaa at position 48 is Leu, Ser, Cys, Arg, Ile, His, Phe, Glu, Lys, Thr, Ala, Met, Val or Asn;
Xaa at position 49 is Met, Arg, Ala, Gly, Pro, Asn, His, or Asp;
Xaa at position 50 is Glu, Leu, Thr, Asp, Tyr, Lys, Asn, Ser, Ala, Ile, Val, His, Phe, Met or Gln;
Xaa at position 51 is Asn, Arg, Met, Pro, Ser, Thr, or His;
Xaa at position 52 is Asn, His, Arg, Leu, Gly, Ser, or Thr;
Xaa at position 53 is Leu, Thr, Ala, Gly, Glu, Pro, Lys, Ser, or Met;
Xaa at position 54 is Arg, Asp, Ile, Ser, Val, Thr, Gln, Asn, Lys, His, Ala or Leu;
Xaa at position 55 is Arg, Thr, Val, Ser, Leu, or Gly;
Xaa at position 56 is Pro, Gly, Cys, Ser, Gln, Glu, Arg, His, Thr, Ala, Tyr, Phe, Leu, Val or Lys;
Xaa at position 57 is Asn or Gly;
Xaa at position 58 is Leu, Ser, Asp, Arg, Gln, Val, or Cys;
Xaa at position 59 is Glu, Tyr, His, Leu, Pro, or Arg;
Xaa at position 60 is Ala, Ser, Pro, Tyr, Asn, or Thr;
Xaa at position 61 is Phe, Asn, Glu, Pro, Lys, Arg, or Ser;
Xaa at position 62 is Asn, His, Val, Arg, Pro, Thr, Asp, or Ile;
Xaa at position 63 is Arg, Tyr, Trp, Lys, Ser, His, Pro, or Val;
Xaa at position 64 is Ala, Asn, Pro, Ser, or Lys;
Xaa at position 65 is Val, Thr, Pro, His, Leu, Phe, or Ser;
Xaa at position 66 is Lys, Ile, Arg, Val, Asn, Glu, or Ser;
Xaa at position 67 is Ser, Ala, Phe, Val, Gly, Asn, Ile, Pro, or His;
Xaa at position 68 is Leu, Val, Trp, Ser, Ile, Phe, Thr, or His;
Xaa at position 69 is Gln, Ala, Pro, Thr, Glu, Arg, Trp, Gly, or Leu;
Xaa at position 70 is Asn, Leu, Val, Trp, Pro, or Ala;
Xaa at position 71 is Ala, Met, Leu, Pro, Arg, Glu, Thr, Gln, Trp, or Asn;
Xaa at position 72 is Ser, Glu, Met, Ala, His, Asn, Arg, or Asp;
Xaa at position 73 is Ala, Glu, Asp, Leu, Ser, Gly, Thr, or Arg;
Xaa at position 74 is Ile, Met, Thr, Pro, Arg, Gly, Ala;
Xaa at position 75 is Glu, Lys, Gly, Asp, Pro, Trp, Arg, Ser, Gln, or Leu;
Xaa at position 76 is Ser, Val, Ala, Asn, Trp, Glu, Pro, Gly, or Asp;
Xaa at position 77 is Ile, Ser, Arg, Thr, or Leu;
Xaa at position 78 is Leu, Ala, Ser, Glu, Phe, Gly, or Arg;
Xaa at position 79 is Lys, Thr, Asn, Met, Arg, Ile, Gly, or Asp;
Xaa at position 80 is Asn, Trp, Val, Gly, Thr, Leu, Glu, or Arg;
Xaa at position 81 is Leu, Gln, Gly, Ala, Trp, Arg, Val, or Lys;
Xaa at position 82 is Leu, Gln, Lys, Trp, Arg, Asp, Glu, Asn, His, Thr, Ser, Ala, Tyr, Phe, Ile, Met or Val;
Xaa at position 83 is Pro, Ala, Thr, Trp, Arg, or Met;
Xaa at position 84 is Cys, Glu, Gly, Arg, Met, or Val;
Xaa at position 85 is Leu, Asn, Val, or Gln;
Xaa at position 86 is Pro, Cys, Arg, Ala, or Lys;
Xaa at position 87 is Leu, Ser, Trp, or Gly;
Xaa at position 88 is Ala, Lys, Arg, Val, or Trp;
Xaa at position 89 is Thr, Asp, Cys, Leu, Val, Glu, His, Asn, or Ser;
Xaa at position 90 is Ala, Pro, Ser, Thr, Gly, Asp, Ile, or Met;
Xaa at position 91 is Ala, Pro, Ser, Thr, Phe, Leu, Asp, or His;
Xaa at position 92 is Pro, Phe, Arg, Ser, Lys, His, Ala, Gly, Ile or Leu;
Xaa at position 93 is Thr, Asp, Ser, Asn, Pro, Ala, Leu, or Arg;
Xaa at position 94 is Arg, Ile, Ser, Glu, Leu, Val, Gln, Lys, His, Ala, or Pro;
Xaa at position 95 is His, Gln, Pro, Arg, Val, Leu, Gly, Thr, Asn, Lys, Ser, Ala, Trp, Phe, Ile, or Tyr;
Xaa at position 96 is Pro, Lys, Tyr, Gly, Ile, or Thr;
Xaa at position 97 is Ile, Val, Lys, Ala, or Asn;
Xaa at position 98 is His, Ile, Asn, Leu, Asp, Ala, Thr, Glu, Gln, Ser, Phe, Met, Val, Lys, Arg, Tyr or Pro;
Xaa at position 99 is Ile, Leu, Arg, Asp, Val, Pro, Gln, Gly, Ser, Phe, or His;
Xaa at position 100 is Lys, Tyr, Leu, His, Arg, Ile, Ser, Gln, or Pro;
Xaa at position 101 is Asp, Pro, Met, Lys, His, Thr, Val, Tyr, Glu, Asn, Ser, Ala, Gly, Ile, Leu, or Gln;
Xaa at position 102 is Gly, Leu, Glu, Lys, Ser, Tyr, or Pro;
Xaa at position 103 is Asp, or Ser;
Xaa at position 104 is Trp, Val, Cys, Tyr, Thr, Met, Pro, Leu, Gln, Lys, Ala, Phe, or Gly;
Xaa at position 105 is Asn, Pro, Ala, Phe, Ser, Trp, Gln, Tyr, Leu, Lys, Ile, Asp, or His;
Xaa at position 106 is Glu, Ser, Ala, Lys, Thr, Ile, Gly, or Pro;
Xaa at position 108 is Arg, Lys, Asp, Leu, Thr, Ile, Gln, His, Ser, Ala or Pro;
Xaa at position 109 is Arg, Thr, Pro, Glu, Tyr, Leu, Ser, or Gly;
Xaa at position 110 is Lys, Ala, Asn, Thr, Leu, Arg, Gln, His, Glu, Ser, or Trp;
Xaa at position 111 is Leu, Ile, Arg, Asp, or Met;
Xaa at position 112 is Thr, Val, Gln, Tyr, Glu, His, Ser, or Phe;
Xaa at position 113 is Phe, Ser, Cys, His, Gly, Trp, Tyr, Asp, Lys, Leu, Ile, Val or Asn;
Xaa at position 114 is Tyr, Cys, His, Ser, Trp, Arg, or Leu;
Xaa at position 115 is Leu, Asn, Val, Pro, Arg, Ala, His, Thr, Trp, or Met;
Xaa at position 116 is Lys, Leu, Pro, Thr, Met, Asp, Val, Glu, Arg, Trp, Ser, Asn, His, Ala, Tyr, Phe, Gln, or Ile;
Xaa at position 117 is Thr, Ser, Asn, Ile, Trp, Lys, or Pro;
Xaa at position 118 is Leu, Ser, Pro, Ala, Glu, Cys, Asp, or Tyr;
Xaa at position 119 is Glu, Ser, Lys, Pro, Leu, Thr, Tyr, or Arg;
Xaa at position 120 is Asn, Ala, Pro, Leu, His, Val, or Gln;
Xaa at position 121 is Ala, Ser, Ile, Asn, Pro, Lys, Asp, or Gly;
Xaa at position 122 is Gln, Ser, Met, Trp, Arg, Phe, Pro, His, Ile, Tyr, or Cys;
Xaa at position 123 is Ala, Met, Glu, His, Ser, Pro, Tyr, or Leu;
   wherein from 1 to 14 amino acids can be deleted from the N-terminus and/or from 1 to 15 amino acids can be deleted from the C-terminus of said human interleukin-3 mutant polypeptide; and wherein from 4 to 44 of the amino acids designated by Xaa are different from the corresponding amino acids of native (1-133) human interleukin-3;
   R₂ is a hematopoietic growth factor;
   L is a linker capable of Linking R₁ to R₂; and said chimera protein can additionally be immediately preceded by (methionine⁻¹), (alanine⁻¹), or (methionine⁻², alanine⁻¹); and
   (b) harvesting said cultured stem cells.

Additionally, the present invention encompasses methods of ex-vivo expansion of stem cells comprising the steps of (a) separating stem cells from a mixed population of cells; (b) culturing said separated stem cells with a growth medium comprising a chimera protein; (c) harvesting said cultured cells.

The present invention includes methods for treatment of a patient having a hematopoietic disorder, comprising the steps of; (a) removing stem cells from said patient or a blood donor; (b) culturing said stem cells with a growth medium comprising a chimera protein; (c) harvesting said cultured cells; and (d) transplanting said cultured cells into said patient.

The present invention also includes methods for treatment of a patient having a hematopoietic disorder, comprising the steps of; (a) removing stem cells from said patient or a blood donor; (b) separating stem cells from a mixed population of cells; (c) culturing said separated stem cells with a growth medium comprising a chimera protein; (d) harvesting said cultured cells; and (e) transplanting said cultured cells into said patient.

It is also envisioned that a patient could be given a hematopoietic growth factor, preferably a early acting factor, prior to removing stem cells for ex-vivo expansion to increase the number of early progenitors. It is also envisioned that a portion of the stem cells removed from a patient could be frozen and transplanted with the expanded stem cells to provide more early progenitors.

It is envisioned that the present invention includes methods of human gene therapy, comprising the steps of; (a) removing stem cells from a patient or blood donor; (b) culturing said stem cells with a selected growth medium comprising a chimera protein; (c) introducing DNA into said cultured cells; (d) harvesting said transduced cells; and (e) transplanting said transduced cells into said patient.

It is also envisioned that the present invention includes methods of human gene therapy, comprising the steps of; (a) removing stem cells from a patient or blood donor; (b) separating said stem cells from a mixed population of cells; (c) culturing said separated stem cells with a selected growth medium comprising a chimera protein; (d) introducing DNA into said cultured cells; (e) harvesting said transduced cells; and (f) transplanting said transduced cells into said patient.

It is also intended that the present invention includes methods of ex vivo expansion of hematopoietic cells, mwthods of expanding hematopoietic cells for gene therapy and methods of treating a patient using the expanded cells using the chimeric proteins of the present invention with other hematopoietic growth factors. A non-exclusive list of other appropriate hematopoietic growth factors, colony stimulating factors, cytokines, lymphokines, hematopoietic growth factors and interleukins for simultaneous or serial co-administration with the polypeptides of the present invention includes GM-CSF, CSF-1, G-CSF, G-CSF Ser¹⁷, c-mpl ligand (MGDF or TPO), c-mpl receptor agonists disclosed in PCT/US96/15938, M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, B-cell growth factor, B-cell differentiation factor and eosinophil differentiation factor, stem cell factor (SCF) also known as steel factor or c-kit ligand, multi-functional hematopoietic receptor agonists disclosed in PCT/US96/15774, or combinations thereof.

### BRIEF DESCRIPTION OF THE DRAWINGS

FIG. 1 is the human IL-3 gene for E. coli expression (pMON5873), encoding the polypeptide sequence of natural (wild type) human IL-3 (SEQ ID NO:49), plus an initiator methionine, as expressed in E. coli, with the amino acids numbered from the N-terminus of the natural hIL-3.

FIG. 2 shows the bioactivity, as measured in the methylcellulose assay, of the polypeptide chimera pMON3988.

FIG. 3 shows the bioactivity, as measured in the methylcellulose assay, of the polypeptide chimeras pMON3987 and pMON26430, pMON3995 and pMON26415.

FIG. 4 shows the bioactivity, as measured in the methylcellulose assay, of the polypeptide chimera pMON26425.

FIG. 5 shows the bioactivity, as measured in the methylcellulose assay, of the polypeptide chimeras pMON26406 and pMON26433.

FIG. 6 shows the bioactivity, as measured in the methylcellulose assay, of the polypeptide chimeras pMON26431 and pMON26427.

### DETAILED DESCRIPTION OF THE INVENTION

The present invention encompasses methods of ex-vivo expansion of hematopoietic cells using a chimera protein comprising a recombinant human interleukin-3 (hIL-3) variants or mutant proteins (muteins) joined with or without a linker to a second IL-3 mutein, IL-3 or a second factor including but not limited to colony stimulating factors, cytokines, lymphokines, interleukins, hematopoietic growth factors or IL-3 variants. This invention encompasses the ex-vivo expansion use of these mixed function hematopoietic growth factors (chimera protein) formed from covalently linked polypeptides, each of which may act through a different and specific cell receptor to initiate complementary biological activities.

Hematopoiesis requires a complex series of cellular events in which stem cells generate continuously into large populations of maturing cells in all major lineages. There are currently at least 20 known regulators with hematopoietic proliferative activity. Most of these proliferative regulators can stimulate one or another type of colony formation in vitro, the precise pattern of colony formation stimulated by each regulator is quite distinctive. No two regulators stimulate exactly the same pattern of colony formation, as evaluated by colony numbers or, more importantly, by the lineage and maturation pattern of the cells making up the developing colonies. Proliferative responses can most readily be analyzed in simplified in vitro culture systems. Three quite different parameters can be distinguished: alteration in colony size, alteration in colony numbers and cell lineage. Two or more factors may act on the progenitor cell, inducing the formation of larger number of progeny thereby increasing the colony size. Two or more factors may allow increased number of progenitor cells to proliferate either because distinct subsets of progenitors cells exist that respond exclusively to one factor or because some progenitors require stimulation by two or more factors before being able to respond. Activation of additional receptors on a cell by the use of two or more factors is likely to enhance the mitotic signal because of coalescence of initially differing signal pathways into a common final pathway reaching the nucleus (Metcalf, Nature 339:27, 1989). Other mechanisms could explain synergy. For example, if one signaling pathway is limited by an intermediate activation of an additional signaling pathway by a second factor may result in a superadditive response. In some cases, activation of one receptor type can induce a enhanced expression of other receptors (Metcalf, Blood 82(12):3515-3523 1993). Two or more factors may result in a different pattern of cell lineages then from a single factor. The use of chimera molecules may have the potential clinical advantage resulting from a proliferative response that is not possible by any single factor.

Hematopoietic and other growth factors can be grouped in to two distinct families of related receptors: (1) tyrosine kinase receptors, including those for epidermal growth factor, M-CSF (Sherr, 1990) and SCF (Yarden et al., EMBO J 6:3341, 1987): and (2) hematopoietic receptors, not containing a tyrosine kinase domain, but exhibiting obvious homology in their extracellular domain (Bazan, Proc. Natl. Acad. Sci. U.S.A. 87(18):6934-8 1990). Included in this later group are erythropoietin (EPO) (D'Andrea et al., Cell 57:277 1989), GM-CSF (Gearing et al., EMBO J 8:3667 1989), IL-3 (Kitamura et al., Cell 66:1165 1991), G-CSF (Fukunaga et al., J. Biol. Chem. 265(23):14008-15 1990), IL-4 (Harada et al., 1990), IL-5 (Takaki et al., EMBO J 9:4367 1990), IL-6 (Yamasaki et al., Science 241:825 1988), IL-7 (Goodwin et al., Cell 60(6):941-51 1990), LIF (Gearing et al., EMBO J 10:2839 1991) and IL-2 (Cosman et al., 1987). Most of the later group of receptors exists in high-affinity form as a heterodimers. After ligand binding, the specific α-chains become associated with at least one other receptor chain (β-chain, γ-chain). Many of these factors share a common receptor subunit. The α-chains for GM-CSF, IL-3 and IL-5 share the same β-chain (Kitamura et al., Cell 66:1165 1991, Takaki et al., EMBO. J. 10(10):2833-8 1991) and receptor complexes for IL-6, LIF and IL-11 share a common β-chain (gp130) (Taga et al., Cell 58(3):573-81 1989; Gearing et al., EMBO J 10:2839 1992). The receptor complexes of IL-2, IL-4 and IL-7 share a common γ-chain (Kondo et al., Science 262:1874 1993; Russell et al., Science 262:1880 1993; Noguchi et al., Science 262:1877 1993).

The ex-vivo expansion methods of the present invention use chimera proteins of the formula selected from the group consisting of
R₁-L-R₂, R₂-L-R₁, R₁-R₂, R₂-R₁, R₁-L-R₁ and R₁-R₁
where R1 is a hIL-3 variant which contains multiple amino acid substitutions and which may have portions of the hIL-3 molecule deleted as is disclosed in WO 94/12638, R2 is a hematopoietic growth factor with a different but complementary activity. By complementary activity is meant activity which enhances or changes the response to another cell modulator. The R1 polypeptide is joined either directly or through a linker segment to the R2 polypeptide. The term “directly” defines chimeras in which the polypeptides are joined without a peptide linker. Thus L represents a chemical bound or polypeptide segment to which both R1 and R2 are joined in frame, most commonly L is a linear peptide to which R1 and R2 are bound by amide bonds linking the carboxy terminus of R1 to the amino terminus of L and carboxy terminus of L to the amino terminus of R2. By “joined in frame” is meant that there is no translation termination or disruption between the reading frames of the DNA sequence encoding R1 and R2. A non-exclusive list of other growth factors, colony stimulating factors, cytokines, lymphokines, interleukins, and hematopoietic growth factors within the definition of R2, which can be joined to a hIL-3 variant of the present invention include GM-CSF, CSF-1, G-CSF, G-CSF Ser¹⁷, c-mpl ligand (MGDF or TPO), M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor and stem cell factor (SCF) also known as steel factor or c-kit ligand. Additionally, this invention encompasses the use of modified R2 molecules or mutated or modified DNA sequences encoding these R2 molecules. The present invention also includes chimera molecules in which R2 is a hIL-3 variant which means an IL-3 in which has amino acid substitutions and which may have portions of the hIL-3 molecule deleted such as what is disclosed in WO 94/12638 and WO 94/12639 as well as other variants known in the art.

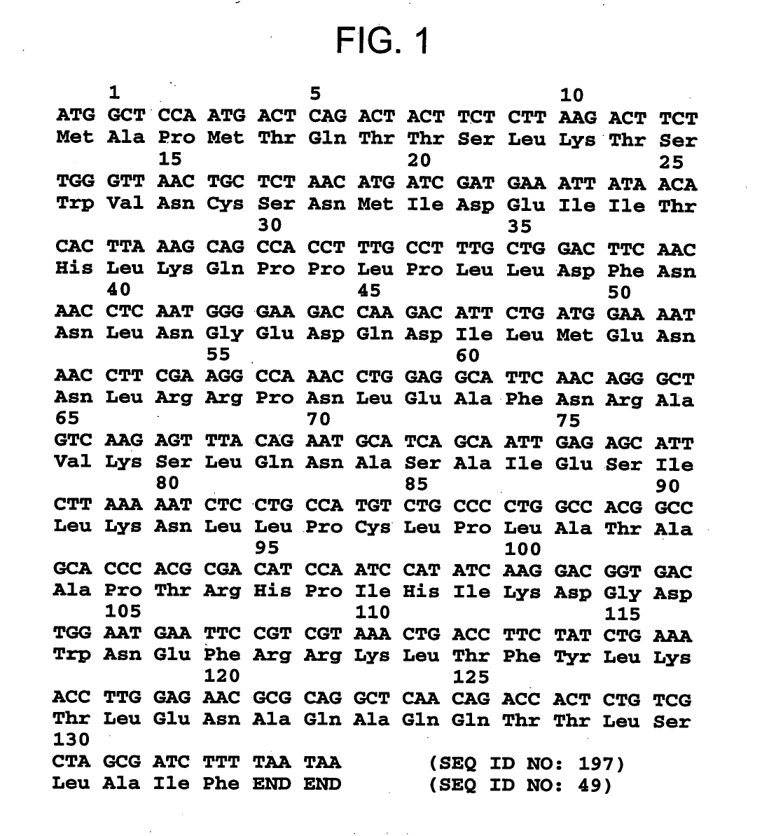

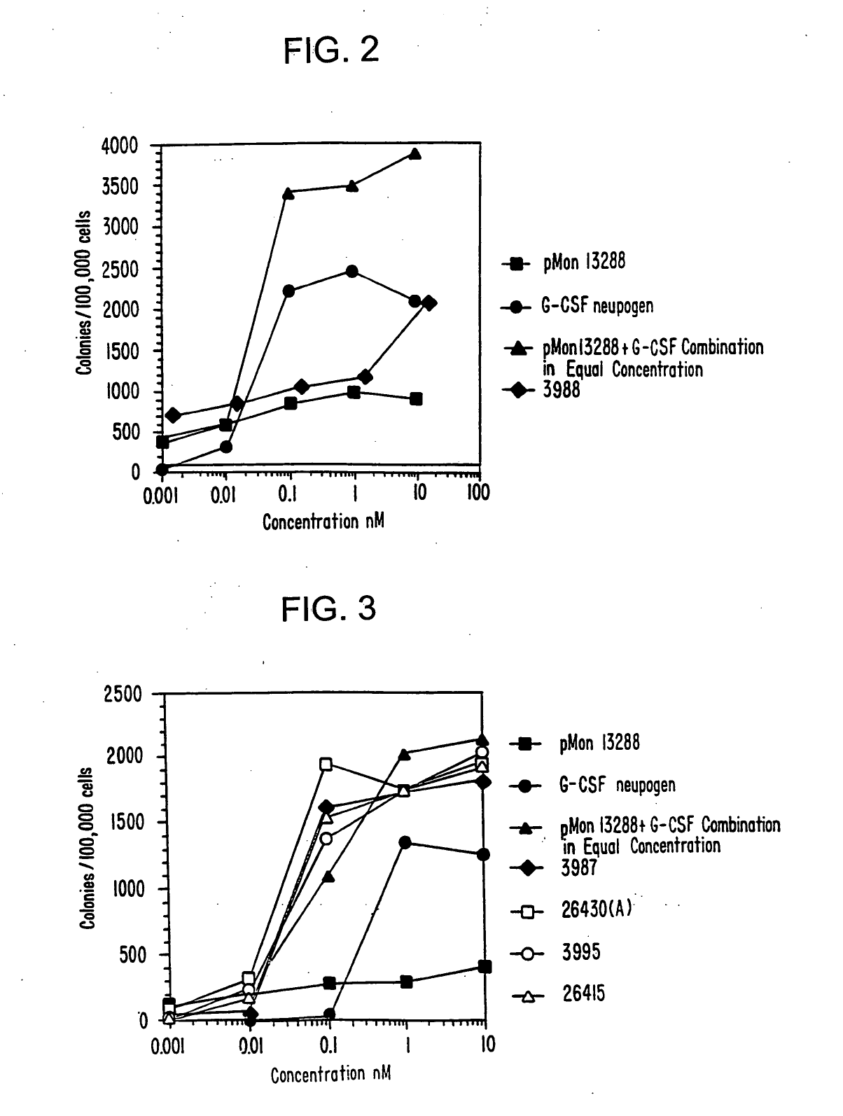

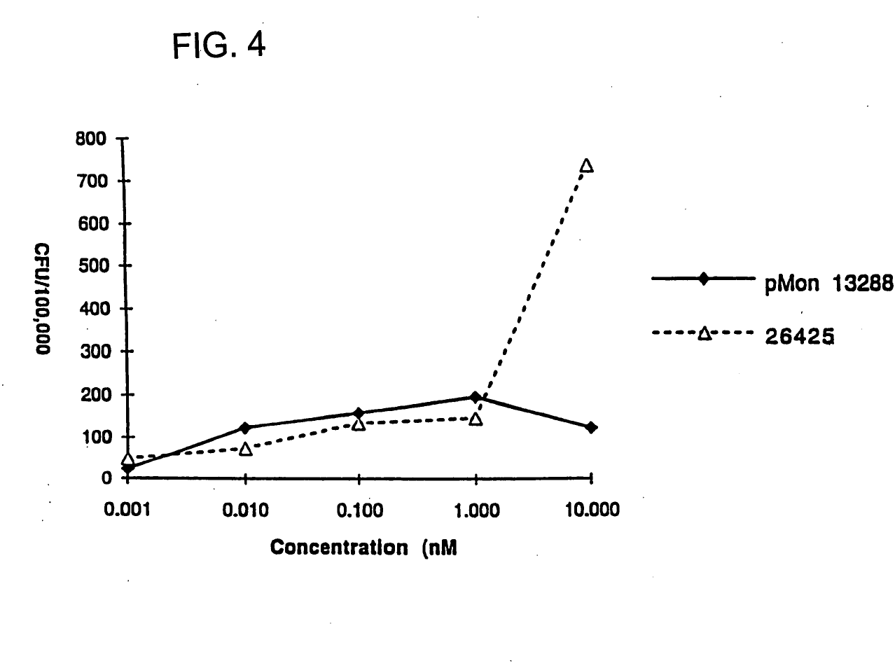

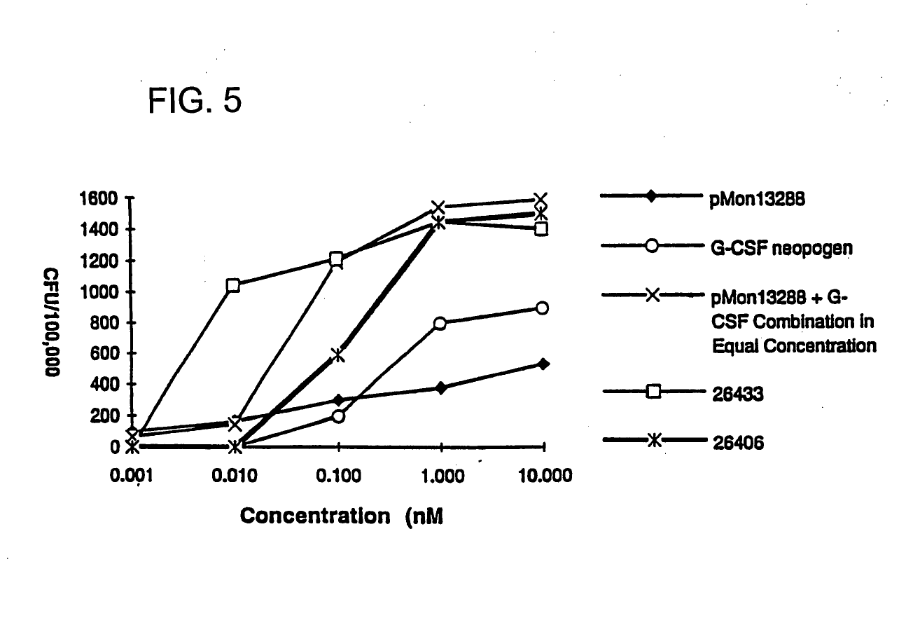

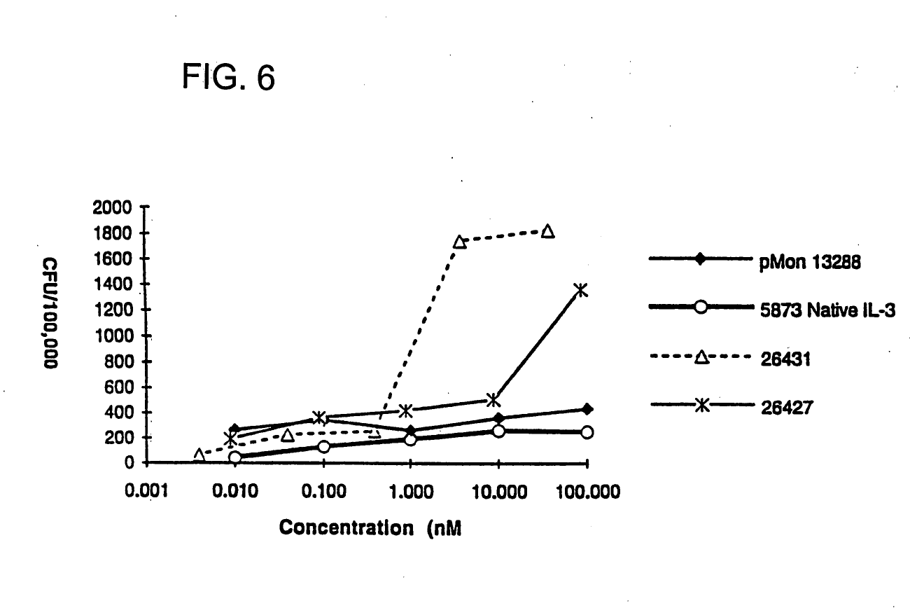

As used herein human interleukin-3 corresponds to the amino acid sequence (1-133) as depicted in FIG. 1 and (15-125) hIL-3 corresponds to the 15 to 125 amino acid sequence of the hIL-3 polypeptide. Naturally occurring variants of hIL-3 polypeptide amino acids are also included in the present invention (for example, the allele in which proline rather than serine is at position 8 in the hIL-3 polypeptide sequence) as are variant hIL-3 molecules which are modified post-translationally (e.g. glycosylation).

“Mutant amino acid sequence,” “mutant protein” or “mutant polypeptide” refers to a polypeptide having an amino acid sequence which varies from a native sequence or is encoded by a nucleotide sequence intentionally made variant from a native sequence. “Mutant protein,” “variant protein” or “mutein” means a protein comprising a mutant amino acid sequence and includes polypeptides which differ from the amino acid sequence of native hIL-3 due to amino acid deletions, substitutions, or both. “Native sequence” refers to an amino acid or nucleic acid sequence which is identical to a wild-type or native form of a gene or protein.

Human IL-3 can be characterized by its ability to stimulate colony formation by human hematopoietic progenitor cells. The colonies formed include erythroid, granulocyte, megakaryocyte, granulocytic macrophages and mixtures thereof. Human IL-3 has demonstrated an ability to restore bone marrow function and peripheral blood cell populations to therapeutically beneficial levels in studies performed initially in primates and subsequently in humans (Gillio, A. P., et al. J. Clin. Invest. 85: 1560 (1990); Ganser, A., et al. Blood 76: 666 (1990); Falk, S., et al. Hematopathology 95: 355 (1991). Additional activities of hIL-3 include the ability to stimulate leukocyte migration and chemotaxis; the ability to prime human leukocytes to produce high levels of inflammatory mediators like leukotrienes and histamine; the ability to induce cell surface expression of molecules needed for leukocyte adhesion; and the ability to trigger dermal inflammatory responses and fever. Other IL-3-like properties are the interaction with early multipotential stem cells, the sustaining of the growth of pluripotent precursor cells, the ability to stimulate chronic myelogenous leukemia (CML) cell proliferation, the stimulation of proliferation of mast cells, the ability to support the growth of various factor-dependent cell lines, and the ability to trigger immature bone marrow cell progenitors. Other biological properties of IL-3 have been disclosed in the art. Many or all of these biological activities of hIL-3 involve signal transduction and high affinity receptor binding.

Biological activity of hIL-3 and hIL-3 chimera proteins of the present invention is determined by DNA synthesis by human acute myelogenous leukemia cells (AML). The factor-dependent cell line AML 193 was adapted for use in testing biological activity. The biological activity of hIL-3 and hIL-3 chimera proteins of the present invention is also determined by counting the colony forming units in a bone marrow assay.

Other in vitro cell based assays may also be useful to determine the activity of the chimera molecules depending on the hematopoietic growth factors that comprise the chimera. The following are examples of other useful assays.
TF-1 proliferation assay: The TF-1 cell line was derived from bone marrow of a patient with erythroleukemia (Kitamura et al., J. Cell Physiol. 140:323-334, 1989). TF-1 cells respond to IL-3, GM-CSF, EPO and IL-5.
32D proliferation assay: 32D is a murine IL-3 dependent cell line which does not respond to human IL-3 but does respond to human G-CSF which is not species restricted.
T1165 proliferation assay: T1165 cells are a IL-6 dependent murine cell line (Nordan et al., Science 233:566, 1986) which respond to IL-6 and IL-11.
Human Plasma Clot meg-CSF Assay: Used to assay megakaryocyte colony formation activity (Mazur et al., Blood 57:277-286 1981).

Compounds of this invention are preferably made by genetic engineering techniques now standard in the art U.S. Pat. No. 4,935,233 and Sambrook et al., “Molecular Cloning. A Laboratory Manual”, Cold Spring Harbor Laboratory, 1989. One method of creating the preferred hIL-3 (15-125) mutant genes is cassette mutagenesis (Wells, et al. Gene, 34:315-323, 1985) in which a portion of the coding sequence of hIL-3 in a plasmid is replaced with synthetic oligonucleotides that encode the desired amino acid substitutions in a portion of the gene between two restriction sites. In a similar manner amino acid substitutions could be made in the full-length hIL-3 gene, or genes encoding variants of hIL-3 in which from 1 to 14 amino acids have been deleted from the N-terminus and/or from 1 to 15 amino acids have been deleted from the C-terminus. When properly assembled these oligonucleotides would encode hIL-3 variants with the desired amino acid substitutions and/or deletions from the N-terminus and/or C-terminus. These and other mutations could be created by those skilled in the art by other mutagenesis methods including; oligonucleotide-directed mutagenesis (Zoller and Smith Nucleic Acid Research, 10:6487-6500, 1982; Zoller and Smith Methods in Enzymology, 100:468-500, 1983; Zoller and Smith DNA, 3: 479, 1984 Smith M. Ann. Rev. Genet., 19:423-462, 1985; Kunkel Proc. Natl. Acad. Sci. USA, 82: 488-492, 1985, Taylor, et al. Nucl. Acids Res., 13:8764-8785 (1985), Deng and Nickoloff, Anal-Biochem 200:81-88, 1992) or polymerase chain reaction (PCR) techniques (Saiki, Science 230:1350-1354, 1985).

Additional details about recombinant techniques for construction of DNA sequences that encode the chimera proteins, plasmid DNA vectors for use in the expression of these novel chimera molecules, methods for producing the chimera molecules in bacterial cells, mammalian cells, or insect cells and the in-vitro and in-vivo activity of the chimera proteins can be found in WO 95/21254. It is understood that the chimera molecules of the present invention, used for the ex-vivo expansion of hematopoietic cells, can be made by other methods known to those skilled in the art.

Hematopoietic cells that have been expanded ex-vivo using the chimera molecules of the present invention may be useful in the treatment of diseases characterized by a decreased levels of either myeloid, erythroid, lymphoid, or megakaryocyte cells of the hematopoietic system or combinations thereof. In addition, they may be used to activate mature myeloid and/or lymphoid cells. Among conditions susceptible to treatment with hematopoietic cells that have been expanded ex-vivo using the chimera proteins of the present invention is leukopenia, a reduction in the number of circulating leukocytes (white cells) in the peripheral blood. Leukopenia may be induced by exposure to certain viruses or to radiation. It is often a side effect of various forms of cancer therapy, e.g., exposure to chemotherapeutic drugs, radiation and of infection or hemorrhage. Therapeutic treatment of leukopenia with these chimera molecules of the present invention may avoid undesirable side effects caused by treatment with presently available drugs.

Hematopoietic cells that have been expanded ex-vivo using the chimera molecules of the present invention may be useful in the treatment of neutropenia and, for example, in the treatment of such conditions as aplastic anemia, cyclic neutropenia, idiopathic neutropenia, Chediak-Higashi syndrome, systemic lupus erythematosus (SLE), leukemia, myelodysplastic syndrome and myelofibrosis.

Hematopoietic cells that have been expanded ex-vivo using the chimera molecule of the present invention may be useful in the treatment or prevention of thrombocytopenia. Currently the only therapy for thrombocytopenia is platelet transfusions which are costly and carry the significant risks of infection (HIV, HBV) and alloimunization. Treatment involving the transplantation of the hematopoietic cells that have been expanded ex-vivo using chimera proteins of the present invention into a patient, may alleviate or diminish the need for platelet transfusions. Severe thrombocytopenia may result from genetic defects such as Fanconi's Anemia, Wiscott-Aldrich, or May-Hegglin syndromes. Acquired thrombocytopenia may result from auto- or allo-antibodies as in Immune Thrombocytopenia Purpura, Systemic Lupus Erythromatosis, hemolytic anemia, or fetal maternal incompatibility. In addition, splenomegaly, disseminated intravascular coagulation, thrombotic thrombocytopenic purpura, infection or prosthetic heart valves may result in thrombocytopenia. Severe thrombocytopenia may also result from chemotherapy and/or radiation therapy or cancer. Thrombocytopenia may also result from marrow invasion by carcinoma, lymphoma, leukemia or fibrosis.

One aspect of the present invention provides a novel hematopoietic factors for selective ex-vivo expansion of stem cells. The term “stem cell” refers to the totipiotent hematopoietic stem cells as well as early precursors and progenitor cells which can be isolated from bone marrow, spleen or peripheral blood. The term “expanding” refers to the differentiation and proliferation of the cells. The present invention provides a method for selective ex-vivo expansion of stem cells, comprising the steps of; (a) separating stem cells from a mixed population of cells, (b) culturing said separated stem cells with a selected media which contains a chimera protein(s) and (c) harvesting said cultured stems cells.

Stem cells as well as committed progenitor cells destined to become neutrophils, erythrocytes, platelets. etc., may be distinguished from most other cells by the presence or absence of particular progenitor marker antigens, such as CD34, that are present on the surface of these cells and/or by morphological characteristics. The phenotype for a highly enriched human stem cell fraction is reported as CD34+, Thy-1+ and lin-, but it is to be understood that the present invention is not limited to the expansion of this stem cell population. The CD34+ enriched human stem cell fraction can be separated by a number of reported methods, including affinity columns or beads, magnetic beads or flow cytometry using antibodies directed to surface antigens such as the CD34+. Further, physical separation methods such as counterflow elutriation may be used to enrich hematopoietic progenitors. The CD34+ progenitors are heterogeneous, and may be divided into several subpopulations characterized by the presence or absence of coexpression of different lineage associated cell surface associated molecules. The most immature progenitor cells do not express any known lineage-associated markers, such as HLA-DR or CD38, but they may express CD90 (thy-1). Other surface antigens such as CD33, CD38, CD41, CD71, HLA-DR or c-kit can also be used to selectively isolate hematopoietic progenitors. The separated cells can be incubated in selected medium in a culture flask, sterile bag or in hollow fibers. Various hematopoietic growth factors may be utilized in order to selectively expand cells. Representative factors that have been utilized for ex-vivo expansion of bone marrow include, c-kit ligand, IL-3, G-CSF, GM-CSF, IL-1, IL-6, IL-11, flt-3 ligand or combinations thereof. The proliferation of the stem cells can be monitored by enumerating the number of stem cells and other cells, by standard techniques (e.g. hemacytometer, CFU, LTCIC) or by flow cytometry prior and subsequent to incubation.

Several methods for ex-vivo expansion of stem cells have been reported utilizing a number of selection methods and expansion using various hematopoietic growth factors including c-kit ligand (Brandt et al., Blood 83:1507-1514 (1994), McKenna et al., Blood 86:3413-3420 (1995), IL-3 (Brandt et al., Blood 83:1507-1514 (1994), Sato et al., Blood 82:3600-3609 (1993), G-CSF (Sato et al., Blood 82:3600-3609 (1993), GM-CSF (Sato et al., Blood 82:3600-3609 (1993), IL-1 (Muench et al., Blood 81:3463-3473 (1993), IL-6 (Sato et al., Blood 82:3600-3609 (1993), IL-11 (Lemoli et al., Exp. Hem. 21:1668-1672 (1993), Sato et al., Blood 82:3600-3609 (1993), flt-3 ligand (McKenna et al., Blood 86:3413-3420 (1995) and/or combinations thereof (Brandt et al., Blood 83:1507-1514 (1994), Haylock et al., Blood 80:1405-1412 (1992), Koller et al., Biotechnology 11:358-363 (1993), (Lemoli et al., Exp. Hem. 21:1668-1672 (1993), McKenna et al., Blood 86:3413-3420 (1995), Muench et al., Blood 81:3463-3473 (1993), Patchen et al., Biotherapy 7:13-26 (1994), Sato et al., Blood 82:3600-3609 (1993), Smith et al., Exp. Hem. 21:870-877 (1993), Steen et al., Stem Cells 12:214-224 (1994), Tsujino et al., Exp. Hem. 21:1379-1386 (1993). Among the individual hematopoietic growth factors, hIL-3 has been shown to be one of the most potent in expanding peripheral blood CD34+ cells (Sato et al., Blood 82:3600-3609 (1993), Kobayashi et al., Blood 73:1836-1841 (1989). However, no single factor has been shown to be as effective as the combination of multiple factors. The present invention provides methods for ex vivo expansion that utilize molecules that are more effective than IL-3 alone.

Another projected clinical use of growth factors has been in the in vitro activation of hematopoietic progenitors and stem cells for gene therapy. Due to the long life-span of hematopoietic progenitor cells and the distribution of their daughter cells throughout the entire body, hematopoietic progenitor cells are good candidates for ex vivo gene transfection. In order to have the gene of interest incorporated into the genome of the hematopoietic progenitor or stem cell one needs to stimulate cell division and DNA replication. Hematopoietic stem cells cycle at a very low frequency which means that growth factors may be useful to promote gene transduction and thereby enhance the clinical prospects for gene therapy. Potential applications of gene therapy (review Crystal, Science 270:404-410 (1995) include; 1) the treatment of many congenital metabolic disorders and immunodifiencies (Kay and Woo, Trends Genet. 10:253-257 (1994), 2) neurological disorders (Freedmann, Trends Genet. 10:210-214 (1994), 3) cancer (Culver and Blaese, Trends Genet. 10:174-178 (1994) and 4) infectious diseases (Gilboa and Smith, Trends Genet. 10:139-144 (1994). Due to the long life-span of hematopoietic progenitor cells and the distribution of their daughter cells throughout the entire body, hematopoietic progenitor cells are good candidates for ex vivo gene transfection include the treatment of many congenital metabolic disorders and immunodifiencies (Kay and Woo, Trends Genet. 10:253-257 (1994) neurological disorders (Freedmann, Trends Genet. 10:210-214 (1994), cancer (Culver and Blaese, Trends Genet. 10:174-178 (1994) and infectious diseases (Gilboa and Smith, Trends Genet. 10:139-144 (1994).

There are a variety of methods, known to those with skill in the art, for introducing genetic material into a host cell. A number of vectors, both viral and non-viral have been developed for transferring therapeutic genes into primary cells. Viral based vectors include; 1) replication-deficient recombinant retrovirus (Boris-Lawrie and Temin, Curr. Opin. Genet. Dev. 3:102-109 (1993), Boris-Lawrie and Temin, Annal. New York Acad. Sci. 716:59-71 (1994), Miller, Current Top. Microbiol. Immunol. 158:1-24 (1992) and replication-deficient recombinant adenovirus (Berkner, BioTechniques 6:616-629 (1988), Berkner, Current Top. Microbiol. Immunol. 158:39-66 (1992), Brody and Crystal, Annal. New York Acad. Sci. 716:90-103 (1994). Non-viral based vectors include protein/DNA complexes (Cristiano et al., PNAS USA. 90:2122-2126 (1993), Curiel et al., PNAS USA 88:8850-8854 (1991), Curiel, Annal. New York Acad. Sci. 716:36-58 (1994), electroporation and liposome mediated delivery such as cationic liposomes (Farhood et al., Annal. New York Acad. Sci. 716:23-35 (1994).

The present invention provides an improvement to the existing methods of expanding hematopoietic cells, which new genetic material has been introduced, in that it provides methods utilizing chimera proteins that have improved biological activity, including an activity not seen by any single colony stimulation factor and/or physical properties.

Many drugs may cause bone marrow suppression or hematopoietic deficiencies. Examples of such drugs are AZT, DDI, alkylating agents and anti-metabolites used in chemotherapy, antibiotics such as chloramphenicol, penicillin, gancyclovir, daunomycin and sulfa drugs, phenothiazones, tranquilizers such as meprobamate, analgesics such as aminopyrine and dipyrone, anti convulsants such as phenytoin or carbamazepine, antithyroids such as propylthiouracil and methimazole and diuretics. Hematopoietic cells that have been expanded ex-vivo using the chimera molecules of the present invention may be useful in preventing or treating the bone marrow suppression or hematopoietic deficiencies which often occur in patients treated with these drugs.

Hematopoietic deficiencies may also occur as a result of viral, microbial or parasitic infections and as a result of treatment for renal disease or renal failure, e.g., dialysis. Hematopoietic cells that have been expanded ex-vivo using the chimera molecules of the present invention may be useful in treating such hematopoietic deficiency.

Various immunodeficiencies e.g., in T and/or B lymphocytes, or immune disorders, e.g., rheumatoid arthritis, may also be beneficially affected by treatment with hematopoietic cells that have been expanded ex-vivo using the chimera molecules of the present invention. Immunodeficiencies may be the result of viral infections e.g. HTLVI, HTLVII, HTLVIII, severe exposure to radiation, cancer therapy or the result of other medical treatment. The chimera molecules of the present invention may also be employed, alone or in combination with other hematopoietic growth factors, in the treatment of other blood cell deficiencies, including thrombocytopenia (platelet deficiency), or anemia. Other uses for these novel polypeptides are in the treatment of patients recovering from bone marrow transplants.

As indicated above, the therapeutic method may also include co-administration with other human factors. A non-exclusive list of other appropriate hematopoietic growth factors, colony stimulating factors, cytokines, lymphokines, hematopoietic growth factors and interleukins for simultaneous or serial co-administration with the polypeptides of the present invention includes GM-CSF, CSF-1, G-CSF, G-CSF Ser¹⁷, c-mpl ligand (MGDF or TPO), c-mpl receptor agonists disclosed in PCT/US96/15938, M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, B-cell growth factor, B-cell differentiation factor and eosinophil differentiation factor, stem cell factor (SCF) also known as steel factor or c-kit ligand, multi-functional hematopoietic receptor agonists disclosed in PCT/US96/15774, or combinations thereof.

The treatment of hematopoietic deficiency may include removing hematopoietic cell from a patient, culturing the cell in a medium containing the chimera molecules to differentiate and proliferate the cells and returning the cultured cells to the patient following a medical treatment. In addition, hematopoietic cell can be removed from a blood donor, cultured and given to a patient suffering from a hematopoietic disorder.

The present invention is directed to methods of ex-vivo expansion of hematopoietic cells by culturing the cells with a chimeric proteins(s) of the formula:
R₁-L-R₂, R₂-L-R₁, R₁-R₂, R₂-R₁, R₁-L-R₁ and R₁-R₁

wherein R₁ is a human interleukin-3 mutant polypeptide of the Formula:

| Ala Pro Met Thr Gln Thr Thr Ser Leu Lys Thr Ser Trp Val Asn | (SEQ ID NO:1) |
|---|---|
| 1 5 10 15 | |
| Cys Xaa Xaa Xaa Xaa Xaa Xaa Xaa Xaa Xaa Xaa Xaa Xaa Xaa Xaa | |
| 20 25 30 | |
| Xaa Xaa Xaa Xaa Xaa Xaa Xaa Xaa Asn Xaa Xaa Xaa Xaa Xaa Xaa | |
| 35 40 45 | |
| Xaa Xaa Xaa Xaa Xaa Xaa Xaa Xaa Xaa Xaa Xaa Xaa Xaa Xaa Xaa | |
| 50 55 60 | |
| Xaa Xaa Xaa Xaa Xaa Xaa Xaa Xaa Xaa Xaa Xaa Xaa Xaa Xaa Xaa | |
| 65 70 75 | |
| Xaa Xaa Xaa Xaa Xaa Xaa Xaa Xaa Xaa Xaa Xaa Xaa Xaa Xaa Xaa | |
| 80 85 90 | |
| Xaa Xaa Xaa Xaa Xaa Xaa Xaa Xaa Xaa Xaa Xaa Xaa Xaa Xaa Xaa | |
| 95 100 105 | |
| Xaa Phe Xaa Xaa Xaa Xaa Xaa Xaa Xaa Xaa Xaa Xaa Xaa Xaa Xaa | |
| 110 115 120 | |
| Xaa Xaa Xaa Gln Gln Thr Thr Leu Ser Leu Ala Ile Phe | |
| 125 130 | |

wherein
Xaa at position 17 is Ser, Lys, Gly, Asp, Met, Gln, or Arg;
Xaa at position 18 is Asn, His, Leu, Ile, Phe, Arg, or Gln;
Xaa at position 19 is Met, Phe, Ile, Arg, Gly, Ala, or Cys;
Xaa at position 20 is Ile, Cys, Gln, Glu, Arg, Pro, or Ala;
Xaa at position 21 is Asp, Phe, Lys, Arg, Ala, Gly, Glu, Gln, Asn, Thr, Ser or Val;
Xaa at position 22 is Glu, Trp, Pro, Ser, Ala, His, Asp, Asn, Gln, Leu, Val or Gly;
Xaa at position 23 is Ile, Val, Ala, Leu, Gly, Trp, Lys, Phe, Ser, or Arg;
Xaa at position 24 is Ile, Gly, Val, Arg, Ser, Phe, or Leu;
Xaa at position 25 is Thr, His, Gly, Gln, Arg, Pro, or Ala;
Xaa at position 26 is His, Thr, Phe, Gly, Arg, Ala, or Trp;
Xaa at position 27 is Leu, Gly, Arg, Thr, Ser, or Ala;
Xaa at position 28 is Lys, Arg, Leu, Gln, Gly, Pro, Val or Trp;
Xaa at position 29 is Gln, Asn, Leu, Pro, Arg, or Val;
Xaa at position 30 is Pro, His, Thr, Gly, Asp, Gln, Ser, Leu, or Lys;
Xaa at position 31 is Pro, Asp, Gly, Ala, Arg, Leu, or Gln;
Xaa at position 32 is Leu, Val, Arg, Gln, Asn, Gly, Ala, or Glu;
Xaa at position 33 is Pro, Leu, Gln, Ala, Thr, or Glu;
Xaa at position 34 is Leu, Val, Gly, Ser, Lys, Glu, Gln, Thr, Arg, Ala, Phe, Ile or Met;
Xaa at position 35 is Leu, Ala, Gly, Asn, Pro, Gln, or Val;
Xaa at position 36 is Asp, Leu, or Val;
Xaa at position 37 is Phe, Ser, Pro, Trp, or Ile;
Xaa at position 38 is Asn, or Ala;
Xaa at position 40 is Leu, Trp, or Arg;
Xaa at position 41 is Asn, Cys, Arg, Leu, His, Met, or Pro;
Xaa at position 42 is Gly, Asp, Ser, Cys, Asn, Lys, Thr, Leu, Val, Glu, Phe, Tyr, Ile, Met or Ala;
Xaa at position 43 is Glu, Asn, Tyr, Leu, Phe, Asp, Ala, Cys, Gln, Arg, Thr, Gly or Ser;
Xaa at position 44 is Asp, Ser, Leu, Arg, Lys, Thr, Met, Trp, Glu, Asn, Gln, Ala or Pro;
Xaa at position 45 is Gln, Pro, Phe, Val, Met, Leu, Thr, Lys, Trp, Asp, Asn, Arg, Ser, Ala, Ile, Glu or His;
Xaa at position 46 is Asp, Phe, Ser, Thr, Cys, Glu, Asn, Gln, Lys, His, Ala, Tyr, Ile, Val or Gly;
Xaa at position 47 is Ile, Gly, Val, Ser, Arg, Pro, or His;
Xaa at position 48 is Leu, Ser, Cys, Arg, Ile, His, Phe, Glu, Lys, Thr, Ala, Met, Val or Asn;
Xaa at position 49 is Met, Arg, Ala, Gly, Pro, Asn, His, or Asp;
Xaa at position 50 is Glu, Leu, Thr, Asp, Tyr, Lys, Asn, Ser, Ala, Ile, Val, His, Phe, Met or Gln;
Xaa at position 51 is Asn, Arg, Met, Pro, Ser, Thr, or His;
Xaa at position 52 is Asn, His, Arg, Leu, Gly, Ser, or Thr;
Xaa at position 53 is Leu, Thr, Ala, Gly, Glu, Pro, Lys, Ser, or Met;
Xaa at position 54 is Arg, Asp, Ile, Ser, Val, Thr, Gln, Asn, Lys, His, Ala or Leu;
Xaa at position 55 is Arg, Thr, Val, Ser, Leu, or Gly;
Xaa at position 56 is Pro, Gly, Cys, Ser, Gln, Glu, Arg, His, Thr, Ala, Tyr, Phe, Leu, Val or Lys;
Xaa at position 57 is Asn or Gly;
Xaa at position 58 is Leu, Ser, Asp, Arg, Gln, Val, or Cys;
Xaa at position 59 is Glu, Tyr, His, Leu, Pro, or Arg;
Xaa at position 60 is Ala, Ser, Pro, Tyr, Asn, or Thr;
Xaa at position 61 is Phe, Asn, Glu, Pro, Lys, Arg, or Ser;
Xaa at position 62 is Asn, His, Val, Arg, Pro, Thr, Asp, or Ile;
Xaa at position 63 is Arg, Tyr, Trp, Lys, Ser, His, Pro, or Val;
Xaa at position 64 is Ala, Asn, Pro, Ser, or Lys;
Xaa at position 65 is Val, Thr, Pro, His, Leu, Phe, or Ser;
Xaa at position 66 is Lys, Ile, Arg, Val, Asn, Glu, or Ser;
Xaa at position 67 is Ser, Ala, Phe, Val, Gly, Asn, Ile, Pro, or His;
Xaa at position 68 is Leu, Val, Trp, Ser, Ile, Phe, Thr, or His;
Xaa at position 69 is Gln, Ala, Pro, Thr, Glu, Arg, Trp, Gly, or Leu;
Xaa at position 70 is Asn, Leu, Val, Trp, Pro, or Ala;
Xaa at position 71 is Ala, Met, Leu, Pro, Arg, Glu, Thr, Gln, Trp, or Asn;
Xaa at position 72 is Ser, Glu, Met, Ala, His, Asn, Arg, or Asp;
Xaa at position 73 is Ala, Glu, Asp, Leu, Ser, Gly, Thr, or Arg;
Xaa at position 74 is Ile, Met, Thr, Pro, Arg, Gly, Ala;
Xaa at position 75 is Glu, Lys, Gly, Asp, Pro, Trp, Arg, Ser, Gln, or Leu;
Xaa at position 76 is Ser, Val, Ala, Asn, Trp, Glu, Pro, Gly, or Asp;
Xaa at position 77 is Ile, Ser, Arg, Thr, or Leu;
Xaa at position 78 is Leu, Ala, Ser, Glu, Phe, Gly, or Arg;
Xaa at position 79 is Lys, Thr, Asn, Met, Arg, Ile, Gly, or Asp;
Xaa at position 80 is Asn, Trp, Val, Gly, Thr, Leu, Glu, or Arg;
Xaa at position 81 is Leu, Gln, Gly, Ala, Trp, Arg, Val, or Lys;
Xaa at position 82 is Leu, Gln, Lys, Trp, Arg, Asp, Glu, Asn, His, Thr, Ser, Ala, Tyr, Phe, Ile, Met or Val;
Xaa at position 83 is Pro, Ala, Thr, Trp, Arg, or Met;
Xaa at position 84 is Cys, Glu, Gly, Arg, Met, or Val;
Xaa at position 85 is Leu, Asn, Val, or Gln;
Xaa at position 86 is Pro, Cys, Arg, Ala, or Lys;
Xaa at position 87 is Leu, Ser, Trp, or Gly;
Xaa at position 88 is Ala, Lys, Arg, Val, or Trp;
Xaa at position 89 is Thr, Asp, Cys, Leu, Val, Glu, His, Asn, or Ser;
Xaa at position 90 is Ala, Pro, Ser, Thr, Gly, Asp, Ile, or Met;
Xaa at position 91 is Ala, Pro, Ser, Thr, Phe, Leu, Asp, or His;
Xaa at position 92 is Pro, Phe, Arg, Ser, Lys, His, Ala, Gly, Ile or Leu;
Xaa at position 93 is Thr, Asp, Ser, Asn, Pro, Ala, Leu, or Arg;
Xaa at position 94 is Arg, Ile, Ser, Glu, Leu, Val, Gln, Lys, His, Ala, or Pro;
Xaa at position 95 is His, Gln, Pro, Arg, Val, Leu, Gly, Thr, Asn, Lys, Ser, Ala, Trp, Phe, Ile, or Tyr;
Xaa at position 96 is Pro, Lys, Tyr, Gly, Ile, or Thr;
Xaa at position 97 is Ile, Val, Lys, Ala, or Asn;
Xaa at position 98 is His, Ile, Asn, Leu, Asp, Ala, Thr, Glu, Gln, Ser, Phe, Met, Val, Lys, Arg, Tyr or Pro;
Xaa at position 99 is Ile, Leu, Arg, Asp, Val, Pro, Gln, Gly, Ser, Phe, or His;
Xaa at position 100 is Lys, Tyr, Leu, His, Arg, Ile, Ser, Gln, or Pro;
Xaa at position 101 is Asp, Pro, Met, Lys, His, Thr, Val, Tyr, Glu, Asn, Ser, Ala, Gly, Ile, Leu, or Gln;
Xaa at position 102 is Gly, Leu, Glu, Lys, Ser, Tyr, or Pro;
Xaa at position 103 is Asp, or Ser;
Xaa at position 104 is Trp, Val, Cys, Tyr, Thr, Met, Pro, Leu, Gln, Lys, Ala, Phe, or Gly;
Xaa at position 105 is Asn, Pro, Ala, Phe, Ser, Trp, Gln, Tyr, Leu, Lys, Ile, Asp, or His;
Xaa at position 106 is Glu, Ser, Ala, Lys, Thr, Ile, Gly, or Pro;
Xaa at position 108 is Arg, Lys, Asp, Leu, Thr, Ile, Gln, His, Ser, Ala or Pro;
Xaa at position 109 is Arg, Thr, Pro, Glu, Tyr, Leu, Ser, or Gly;
Xaa at position 110 is Lys, Ala, Asn, Thr, Leu, Arg, Gln, His, Glu, Ser, or Trp;
Xaa at position 111 is Leu, Ile, Arg, Asp, or Met;
Xaa at position 112 is Thr, Val, Gln, Tyr, Glu, His, Ser, or Phe;
Xaa at position 113 is Phe, Ser, Cys, His, Gly, Trp, Tyr, Asp, Lys, Leu, Ile, Val or Asn;
Xaa at position 114 is Tyr, Cys, His, Ser, Trp, Arg, or Leu;
Xaa at position 115 is Leu, Asn, Val, Pro, Arg, Ala, His, Thr, Trp, or Met;
Xaa at position 116 is Lys, Leu, Pro, Thr, Met, Asp, Val, Glu, Arg, Trp, Ser, Asn, His, Ala, Tyr, Phe, Gln, or Ile;
Xaa at position 117 is Thr, Ser, Asn, Ile, Trp, Lys, or Pro;
Xaa at position 118 is Leu, Ser, Pro, Ala, Glu, Cys, Asp, or Tyr;
Xaa at position 119 is Glu, Ser, Lys, Pro, Leu, Thr, Tyr, or Arg;
Xaa at position 120 is Asn, Ala, Pro, Leu, His, Val, or Gln;
Xaa at position 121 is Ala, Ser, Ile, Asn, Pro, Lys, Asp, or Gly;
Xaa at position 122 is Gln, Ser, Met, Trp, Arg, Phe, Pro, His, Ile, Tyr, or Cys;
Xaa at position 123 is Ala, Met, Glu, His, Ser, Pro, Tyr, or Leu;
   wherein from 1 to 14 amino acids can be deleted from the N-terminus and/or from 1 to 15 amino acids can be deleted from the C-terminus; and wherein from 4 to 44 of the amino acids designated by Xaa are different from the corresponding amino acids of native (1-133) human interleukin-3;

R₂ is a hematopoietic growth factor;

L is a linker capable of linking R¹ and R²;and said chimera protein can additionally be immediately preceded by (methionine⁻¹), (alanine⁻¹), or (methionine⁻², alanine⁻¹).

In a preferred embodiment, R² is a hematopoietic growth factor selected from the group consisting of GM-CSF, CSF-1, G-CSF, G-CSF Ser¹⁷, c-mpl ligand (MGDF or TPO), M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor and stem cell factor (SCF).

The dosage regimen involved in ex-vivo expansion of hematopoietic cells and methods for treating the above-described conditions will be determined by the attending physician considering various factors which modify the action of drugs, e.g. the condition, body weight, sex and diet of the patient, the severity of any infection, time of administration and other clinical factors. Generally, a dosage regimen may be in the range of 1 ng to 100 ng of non-glycosylated IL-3 chimeric protein per mL of culture medium. This dosage regimen is referenced to a standard level of biological activity which recognizes that native IL-3 generally possesses an EC₅₀ at or about 10 picoMolar to 100 picoMolar in the AML proliferation assay described herein. Therefore, dosages would be adjusted relative to the activity of a given chimera protein vs. the activity of native (reference) IL-3 and it would not be unreasonable to note that dosage regimens may include doses as low as 0.1 ng and as high as 1 milligram per mL of culture medium. In addition, there may exist specific circumstances where dosages of chimera protein would be adjusted higher or lower. When administered with the chimera proteins of the present invention, other hematopoietic growth factors are used in the range of 1 ng to 100 ng per mL of culture medium. The other hematopoietic growth factors could be used as low as 1 pg/mL and as high as 1 mg/mL depending on the chimeria protein used, the various combination of hematopoietic growth factors used and the nature of the expanded hematopoietic cell population that is desired. Other factors that could effect the dosage of the chimera proteins and other hematopoietic growth factors include; co-administration with chemotherapeutic drugs and/or radiation; the use of glycosylated proteins; and various patient-related issues mentioned earlier in this section.

The following examples will illustrate the invention in greater detail although it will be understood that the invention is not limited to these specific examples.

### EXAMPLE 1

Determination of the In Vitro Activity of Chimera Proteins

The protein concentration of the chimera protein can be determined using a sandwich ELISA based on an affinity purified polyclonal antibody. Alternatively the protein concentration can be determined by amino acid composition. The bioactivity of the chimera molecule can be determined in a number of in vitro assays compared with native IL-3, the IL-3 variant or G-CSF alone or together. One such assay is the AML-193 cell proliferation assay. AML-193 cells respond to IL-3 and G-CSF which allows for the combined bioactivity of the IL-3 variant/G-CSF chimera to be determined. In addition other factor dependent cell lines, such as M-NFS-60 (ATCC. CRL 1838) or 32D which are murine IL-3 dependent cell line, may be used. The activity of IL-3 is species specific whereas G-CSF is not, therefor the bioactivity of the G-CSF component of the IL-3 variant/G-CSF chimera can be determined independently. The methylcellulose assay can be used to determine the effect of the IL-3 variant/G-CSF chimera protein on the expansion of the hematopoietic progenitor cells and the pattern of the different types of hematopoietic colonies in vitro. The methylcellulose assay can provide an estimate of precursor frequency since one measures the frequency of progenitors per 100,000 input cells. Long term, stromal dependent cultures have been used to delineate primitive hematopoietic progenitors and stem cells. This assay can be used to determine whether the chimera molecule stimulates the expansion of very primitive progenitors and/or stem cells. In addition, limiting dilution cultures can be performed which will indicate the frequency of primitive progenitors stimulated by the chimera molecule.

Determination of Bioactivity of Chimera Molecules in AML Proliferation Assay

The AML assay is useful for determining the activity of chimera molecules that respond to hIL-3, G-CSF and

The factor-dependent cell line AML 193 was obtained from the American Type Culture Collection (ATCC, Rockville, Md.). This cell line, established from a patient with acute myelogenous leukemia, is a growth factor dependent cell line which displayed enhanced growth in GM-CSF supplemented medium (Lange, B., et al., Blood 70:192, 1987; Valtieri, M., et al., J. Immunol. 138:4042, 1987). The ability of AML 193 cells to proliferate in the presence of human IL-3 has also been documented. (Santoli, D., et al., J. Immunology 139:348, 1987). A cell line variant was used, AML 193 1.3, which was adapted for long term growth in IL-3 by washing out the growth factors and starving the cytokine dependent AML 193 cells for growth factors for 24 hours. The cells are then replated at 1×10⁵ cells/well in a 24 well plate in media containing 100 U/mL IL-3. It took approximately 2 months for the cells to grow rapidly in IL-3. These cells are maintained as AML 193 1.3 thereafter by supplementing tissue culture medium (see below) with human IL-3.

AML 193 1.3 cells are washed 6 times in cold Hanks balanced salt solution (HBSS, Gibco, Grand Island, N.Y.) by centrifuging cell suspensions at 250×g for 10 minutes followed by decantation of the supernatant. Pelleted cells are resuspended in HBSS and the procedure is repeated until six wash cycles are completed. Cells washed six times by this procedure are resuspended in tissue culture medium at a density ranging from 2×10⁵ to 5×10⁵ viable cells/mL. This medium is prepared by supplementing Iscove's modified Dulbecco's Medium (IMDM, Hazelton, Lenexa, Kans.) with albumin, transferrin, lipids and 2-mercaptoethanol. Bovine albumin (Boehringer-Mannheim, Indianapolis, Ind.) is added at 500 μg/mL; human transferrin (Boehringer-Mannheim, Indianapolis, Ind.) is added at 100 μg/mL; soybean lipid (Boehringer-Mannheim, Indianapolis, Ind.) is added at 50 μMg/mL; and 2-mercaptoethanol (Sigma, St. Louis, Mo.) is added at 5×10⁻⁵ M.

Serial dilutions of human interleukin-3 or chimera protein (hIL-3 mutein) are made in triplicate series in tissue culture medium supplemented as stated above in 96 well Costar 3596 tissue culture plates. Each well contained 50 μl of medium containing interleukin-3 or chimera protein once serial dilutions are completed. Control wells contained tissue culture medium alone (negative control). AML 193 1.3 cell suspensions prepared as above are added to each well by pipetting 50 μl (2.5×10⁴ cells) into each well. Tissue culture plates are incubated at 37° C. with 5% CO₂ in humidified air for 3 days. On day 3, 0.5 μCi ³H-thymidine (2 Ci/mM, New England Nuclear, Boston, Mass.) is added in 50 μl of tissue culture medium. Cultures are incubated at 37° C. with 5% CO₂ in humidified air for 18-24 hours. Cellular DNA is harvested onto glass filter mats (Pharmacia LKB, Gaithersburg, Md.) using a TOMTEC cell harvester (TOMTEC, Orange, Conn.) which utilized a water wash cycle followed by a 70% ethanol wash cycle. Filter mats are allowed to air dry and then placed into sample bags to which scintillation fluid (Scintiverse II, Fisher Scientific, St. Louis, Mo. or BetaPlate Scintillation Fluid, Pharmacia LKB, Gaithersburg, Md.) is added. Beta emissions of samples from individual tissue culture wells are counted in a LKB Betaplate model 1205 scintillation counter (Pharmacia LKB, Gaithersburg, Md.) and data is expressed as counts per minute of ³H-thymidine incorporated into cells from each tissue culture well. Activity of each human interleukin-3 preparation or chimera protein preparation is quantitated by measuring cell proliferation (³H-thymidine incorporation) induced by graded concentrations of interleukin-3 or chimera protein. Typically, concentration ranges from 0.05 pM-10⁵ pM are quantitated in these assays. Activity is determined by measuring the dose of interleukin-3 or chimera molecule which provides 50% of maximal proliferation (EC₅₀=0.5×(maximum average counts per minute of ³H-thymidine incorporated per well among triplicate cultures of all concentrations of interleukin-3 tested—background proliferation measured by ³H-thymidine incorporation observed in triplicate cultures lacking interleukin-3). This EC₅₀ value is also equivalent to 1 unit of bioactivity. Every assay is performed with native interleukin-3 as a reference standard so that relative activity levels could be assigned.

Typically, the protein chimeras were tested in a concentration range of 2000 pM to 0.06 pM titrated in serial 2 fold dilutions. Biological activity of the chimera molecules was compared to the following standards as described below.

Protein chimeras comprised in part of G-CSF, pMON3987, pMON3995, pMON3997, pMON26406, pMON26433, pMON26415, pMON26416, and pMON26430, were compared to the dose response curve of equal molar concentrations of hG-CSF and pMON13288 or pMON13416.

Protein chimeras comprised in part of GM-CSF, pMON3989 and pMON3998 were compared to the dose response curve of equal molar concentrations of hGM-CSF and pMON13288. Protein chimeras comprised of dimers of hIL-3 variants, pMON3988, pMON26425, pMON26427, pMON26420, pMON26429 and pMON26431 were compared to the dose response curve of pMON13288 or pMON13416.

Activity for each sample was determined by the concentration which gave 50% of the maximal response by fitting a four-parameter logistic model to the data. It was observed that the upper plateau (maximal response) for the sample and the standard with which it was compared did not differ. Therefore relative potency calculation for each sample was determined from EC50 estimations for the sample and the standard as indicated above. Relative potency (EC50 of standard divided by EC50 of sample) reported in Table 3 is the mean of at least two independent assays unless indicated.

AML 193.1.3 cells proliferate in response to hIL-3, hGM-CSF and hG-CSF.

TABLE 3
AML cell proliferation assay

| | | | AML 193.1.3 |
|---|---|---|---|
| | | | Bioactivity |
| | | | (relative |
| pMON | R₁ | R₂ | potency) |
| pMON3987 | 13288 | G-CSF | 0.35 ± 0.11 |
| pMON3988 | 13288 | 13288 | 0.64 ± 0.13 |
| pMON3989 | 13288 | GM-CSF | 0.6 ± 0.09 |
| pMON3995 | G-CSF | 13288 | 0.41 ± 0.44 |
| pMON3997 | 13288 | G-CSF | 0.26 (n = 1) |
| pMON3998 | 13288 | GM-CSF | 0.21 (n = 1) |
| pMON26406 | 13288 | G-CSF | 0.37 ± 0.30 |
| pMON26433 | G-CSF | 13288 | 0.79 ± 0.35 |
| pMON26415 | 13288 | G-CSF Ser17 | 0.46 ± 0.08 |
| pMON26416 | G-CSF | 13416 | 0.43 ± 0.02 |
| pMON26425 | 13288 | 13288 | 1.32 ± 0.41 |
| pMON26427 | 13288 | 13288 | 1.41 ± 0.91 |
| pMON26420 | 13416 | 13416 | 2.09 ± 0.52 |
| pMON26430 | 13288 | G-CSF | 1.04 ± 0.69 |
| pMON26429 | 13288 | 13288 | 1.88 ± 0.09 |
| pMON26431 | 13288 | 13288 | 0.66 ± 0.26 |

### EXAMPLE 2

Determination of Bioactivity of Chimera Molecules in Methylcellulose Assay

This assays the ability of hematopoietic growth factors to stimulate normal bone marrow cells to produce different types of hematopoietic colonies in vitro (Bradley et al., Aust. Exp. Biol. Med. Sci. 44:287-300 1966; Pluznik et al., J Cell Comp Physiol 66:319-324 1965).

Methods

Approximately 30 mL of fresh, normal, healthy bone marrow aspirate are obtained from individuals. Under sterile conditions samples are diluted 1:5 with a 1×PBS (#14040.059 Life Technologies, Gaithersburg, Md.) solution in a 50 mL conical tube (#25339-50 Corning, Corning Md.). Ficoll (Histopaque 1077 Sigma H-8889) is layered under the diluted sample and centrifuged, 300× g for 30 min. The mononuclear cell band is removed and washed two times in 1×PBS and once with 1% BSA PBS (CellPro Co., Bothel, Wash.). Mononuclear cells are counted and CD34+ cells are selected using the Ceprate LC (CD34) Kit (CellPro Co., Bothel, Wash.) column. This fractionation is performed since all stem and progenitor cells within the bone marrow display CD34 surface antigen.

Cultures are set up in triplicate with a final volume of 1.0 mL in a 35×10 mm petri dish (Nunc#174926). Culture medium is purchased from Terry Fox Labs. (HCC-4230 medium (Terry Fox Labs, Vancouver, B.C., Canada) and erythropoietin (Amgen, Thousands Oaks, Calif.) is added to the culture media. 3,000-10,000 CD34+ cells are added per dish. Native IL-3 and chimera molecules are added to give final concentrations ranging from 0.001 nM 10 nM. Native IL-3 and chimera molecules are supplied in house. G-CSF (Neupogen) is from Amgen. Cultures are resuspended using a 3cc syringe and 1.0 mL is dispensed per dish. Control (baseline response) cultures received no hematopoietic growth factors . Positive control cultures received conditioned media (PHA stimulated human cells; Terry Fox Lab. H2400). Cultures are incubated at 37° C., 5% CO₂ in humidified air.

Hematopoietic colonies which are defined as greater than 50 cells are counted on the day of peak response (days 10-11) using a Nikon inverted phase microscope with a 40× objective combination. Groups of cells containing fewer than 50 cells are referred to as clusters. Alternatively colonies can be identified by spreading the colonies on a slide and stained or they can be picked, resuspended and spun onto cytospin slides for staining.

### EXAMPLE 3

Determination of Bioactivity of Chimera Molecules in Human Cord Blood Hematopoietic Growth Factor Assay

Bone marrow cells are traditionally used for in vitro assays of hematopoietic growth factor activity. However, human bone marrow is not always available, and there is considerable variability between donors. Umbilical cord blood is comparable to bone marrow as a source of hematopoietic stem cells and progenitors (Broxmeyer et al., Proc. Natl. Acad. Sci. USA, 89:4109-4113 1992; Mayani et al., Blood 81:3252-3258 1993). In contrast to bone marrow, cord blood is more readily available on a regular basis. There is also a potential to reduce assay variability by pooling cells obtained fresh from several donors, or to create a bank of cryopreserved cells for this purpose. By modifying the culture conditions, and/or analyzing for lineage specific markers, it should be possible to assay specifically for granulocyte/macrophage colonies (CFU-GM), for megakaryocyte CSF activity, or for high proliferative potential colony forming cell (HPP-CFC) activity.

Methods

Mononuclear cells (MNC) are isolated from cord blood within 24 hr. of collection, using a standard density gradient (1.077 g/mL Histopaque). Cord blood MNC have been further enriched for stem cells and progenitors by several procedures, including immunomagnetic selection for CD14−, CD34+ cells; panning for SBA-, CD34+ fraction using coated flasks from Applied Immune Science (Santa Clara, Calif.); and CD34+ selection using a CellPro (Bothell, Wash.) avidin column. Either freshly isolated or cryopreserved CD34+ cell enriched fractions are used for the assay. Duplicate cultures for each serial dilution of sample (concentration range from 1 pM to 1204 pM) are prepared with 1×104 cells in 1 ml of 0.9% methylcellulose containing medium without additional growth factors (Methocult H4230 from Stem Cell Technologies, Vancouver, BC.). In some experiments, Methocult H4330 containing erythropoietin (EPO) was used instead of Methocult H4230, or Stem Cell Factor (SCF), 50 ng/mL (Biosource International, Camarillo, Calif.) was added. After culturing for 7-9 days, colonies containing >30 cells are counted. In order to rule out subjective bias in scoring, assays are scored blind.

### EXAMPLE 4

Determination of Bioactivity of Chimera Molecules in Megakaryocyte Proliferation Assay

Methods

1. Bone Marrow Proliferation Assay

a. CD34+ Cell Purification:

Between 15-20 mL bone marrow aspirates were obtained from normal allogeneic marrow donors after informed consent. Cells were diluted 1:3 in phosphate buffered saline (PBS, Gibco-BRL), 30 mL were layered over 15 mL Histopaque-1077 (Sigma) and centrifuged for 30 minutes at 300 RCF. The mononuclear interface layer was collected and washed in PBS. CD34+ cells were enriched from the mononuclear cell preparation using an affinity column per manufacturers instructions (CellPro, Inc, Bothell Wash.). After enrichment, the purity of CD34+ cells was 70% on average as determined by using flow cytometric analysis using anti CD34 monoclonal antibody conjugated to fluorescein and anti CD38 conjugated to phycoerythrin (Becton Dickinson, San Jose Calif.).

Cells were resuspended at 40,000 cells/mL in X-Vivo 10 media (Bio-Whittaker, Walkersville, Md.) and 1 mL was plated in 12-well tissue culture plates (Costar). The growth factor rhIL-3 was added at 100 ng/mL (pMON5873) was added to some wells. hIL3 variant, pMON13288, was used at 10 ng/mL or 100 ng/mL. Conditioned media from BHK cells transfected with plasmid encoding c-mpl ligand were tested by addition of 100 μl of supernatant added to 1 mL cultures (approximately a 10% dilution). Cells were incubated at 37° C. for 8-14 days at 5% CO₂ in a 37° C. humidified incubator.

b. Cell Harvest and Analysis:

At the end of the culture period a total cell count was obtained for each condition. For fluorescence analysis and ploidy determination cells were washed in megakaryocyte buffer (MK buffer, 13.6 mM Sodium Citrate, 1 mM Theophylline, 2.2 μm PGE1, 11 mM Glucose, 3% w/v BSA, in PBS, pH 7.4,) (Tomer et al., Blood 70 (6):1735-1742, 1987) resuspended in 500 μl of MK buffer containing anti-CD41a FITC antibody (1:200, AMAC, Westbrook, Me.) and washed in MK buffer. For DNA analysis cells were permeablized in MK buffer containing 0.5% Tween 20 (Fisher, Fair Lawn N.J.)for 20 min. on ice followed by fixation in 0.5% Tween-20 and 1% paraformaldehyde (Fisher Chemical) for 30 minutes followed by incubation in Propidium Iodide (Calbiochem , La Jolla Calif.) (50 μg/mL) with RNA-ase (400 U/mL) in 55% v/v MK buffer (20 mOsm) for 1-2 hours on ice. Cells were analyzed on a FACScan or Vantage flow cytometer (Becton Dickinson, San Jose, Calif.). Green fluorescence (CD41a-FITC) was collected along with linear and log signals for red fluorescence (PI) to determine DNA ploidy. All cells were collected to determine the percent of cells that were CD41+. Data analysis was performed using software by LYSIS (Becton Dickinson, San Jose, Calif.). Percent of cells expressing the CD41 antigen was obtained from flow cytometry analysis(Percent). Absolute (Abs) number of CD41+ cells/mL was calculated by: (Abs)=(Cell Count)*(Percent)/100.

2. Megakaryocyte Fibrin Clot Assay.

CD34+ enriched population were isolated as described above. Cells were suspended at 25,000 cells/mL with/without cytokine(s) in a media consisting of a base Iscoves IMDM media supplemented with 0.3% BSA, 0.4 mg/mL apo-transferrin, 6.67 μM FeCl₂, 25 μg/mL CaCl₂, 25 μg/mL L-asparagine, 500 μg/mL E-amino-n-caproic acid and Penicillin/Streptomycin. Prior to plating into 35 mm plates, thrombin was added (0.25 Units/mL) to initiate clot formation. Cells were incubated at 37° C. for 13 days at 5% CO₂ in a 37° C. humidified incubator.

At the end of the culture period plates were fixed with Methanol:Acetone (1:3), air dried and stored at −200 C until staining. A peroxidase immunocytochemistry staining procedure was used (Zymed, Histostain-SP. San Francisco, Calif.) using a cocktail of primary monoclonal antibodies consisting of anti CD41a, CD42 and CD61. Colonies were counted after staining and classified as negative, CFU-MK (small colonies, 1-2 foci and less that approx. 25 cells), BFU-MK (large, multi-foci colonies with >25 cells) or mixed colonies (mixture of both positive and negative cells.

### EXAMPLE 4

Ex Vivo Expansion of CD34+ Cells from Peripheral Blood Using Chimera Molecules pMON13056 and pMON13148+/−SCF

Flow Cytometry Evaluation

The percentage of CD34+ cells in the thawed peripheral blood cell population was determined by flow cytometry. Cells were removed from the selected cell population and placed into two centrifuge tube and washed once in 9/1% albumin Phosphate buffer (PAB). Twenty microliters of anti-CD34 monoclonal antibody (8G12-FITC) or mouse monoclonal antibody IGG-FITC control was added to the tube. The tubes were incubated for 15 minutes on ice. The cells were washed once with PAB and resuspended in approximately 0.5 mL PAB. Propidium iodide (2 ug/mL) was added to each tube just prior to the analysis on the FACSort or FACScan. Selected cells that contain greater than 80% CD34+ cells were used to initiate the cultures.

On day 12, cultures were harvested and evaluated with CD41A-FITC (a megakaryocyte marker), CD15-FITC and CD11b-PE (early to late neutrophil marker) and CD34 by flow cytometry, using the same processes of preparation and analysis as described above.

Colony Assay Evaluation

Colony assay evaluation was performed on day 0 with 500-1000 selected CD34+ cells per dish and again on day 12 of culture with 5,000-10,000 cultured cells per dish. The cells were added to a colony assay culture tube containing 3 mL of Terry Fox Iscove's based methylcellulose and the following growth factors: 20 ng/mL SCF, 10 U/mL EPO, 300 U/mL GM-CSF, 300 U/mL G-CSF, 30 U/mL IL3 and 40 ng/mL IL6. Two 35 mm tissue culture dishes containing 1 mL were set up. All dishes were incubated at 37° C., 5% carbon dioxide, 5% oxygen and high humidity for 13-15 days. The dishes were scored for myeloid (CFU-GM), erythroid (BFU-E) or mixed myeloid and erythroid colonies (CFU-mix) using a Nikon SMZU stereoscope.

Cell Morphology Evaluation

On day 12 of culture cells were analyzed for cell morphology after Wright-Giemsa staining. Cultured cells were cytocentrifuged onto slides at 1000 rpm for 4 minutes. Each slide contained approximately 10000-20000 cells. Slides were allowed to air dry before staining with 0.5 mL Wright-Giemsa for 1 minutes and 0.5 mL tap water for 1-2 minutes. Slides were cover-slipped and evaluated using a Microstar light microscope. A differential cell count of neutrophils, megakaryocytes and other blood cells was performed.

RESULTS

CD34+ Selection

Studies were performed on CD34+ cells selected using the Isolex™ 300 magnetic Cell Separator from apheresis products from normal donors mobilized with G-CSF. The selected cells were stored in X-VIVO 10+12.5% HSA containing 10% DMSO in liquid nitrogen until required. Cultures were initiated as described in the methods section.

Proliferation Index of Cultures at Day 12

The proliferation index of cultures was calculated by diving the cell concentration at day 5-7 by 5×10⁴ and then multiplying it by the cell concentration at day 12 divided by 1×10⁵. A summary of the proliferation index obtained from these CD34+ cell cultures is shown in Table 4.

Flow Cytometry Evaluation of Neutrophil Precursors

The percentage of neutrophil precursors in the CD34+ cell cultures at day 12 was assessed by flow cytometry using the CD15 marker for early to late neutrophil precursors and the CD11b marker found on late neutrophil precursors determined is shown in Table 4.

Flow Cytometry Evaluation of Megakaryocytes

The percentage of Mks in the CD34+ cell cultures was assessed by flow cytometry using the CD41a marker for megakaryocytes. The percentage of Mks observed in the CD34+ cell cultures is shown in Table 4.

Flow Cytometry Evaluation of CD34+ Cells

The percentage of CD34+ cells present in the cultures at day 12 was determined by flow cytometry. The percentage of CD34+ cells still remaining in the cultures at day 12 ranged from 0.103-19.3%, with no significant difference or patterns observed with the different growth factor combinations.

Total Number of Megakaryocytes Generated in Culture

The total number of megakaryocytes present in each culture is calculated by multiplying the total number of cells at day 12 by the percentage of CD15+ cells and is shown in Table 4.

Colony Forming Unit Granulocyte-Macrophage (CFU-GM) Index

CFU-GM index is calculate by dividing the total number of GM-colonies obtained at day 12 by the number of GM-colonies obtained at day 0. A CFU-GM index of 1 indicates that the number of colonies at day 12 is equivalent to the number of colonies at the start of the culture. A summary of the CFU-GM index for these cultures is shown in Table 4.

Colony Forming Unit (CFU) Index

CFU index is calculated by dividing the total number of colonies (CFU-GM, BFU-E and mixed) obtained at day 12 by the total number of colonies obtained at day 0. A CFU index of 1 indicates that the number of colonies at day 12 is equivalent to the number of colonies at the start of the culture. A summary of the CFU index for these cultures is shown in Table 4.

TABLE 4
Ex-vivo Expansion

| | Growth | Donor | Donor | Donor | Donor | Donor |
|---|---|---|---|---|---|---|
| Assay | Factor | #1 | #2 | #3 | #4 | #5 |
| Proliferation | pMON13056 | 39.4 | 73.4 | ND | 5.7 | 5.7 |
| Index of CD34+ | pMON13056 + SCF | 135 | 206 | 37.4 | 17.4 | 6.4 |
| Cell Cultures at | pMON13148 | 21.4 | 23.8 | ND | ND | ND |
| Day 12 | pMON13148 + SCF | 88.1 | 117.7 | ND | ND | ND |
| | native | 9 | 4.1 | 10.7 | 1 | 1.4 |
| | hIL-3 | | | | | |
| | native | 70.5 | 61.3 | 62.3 | 22.6 | 12.2 |
| | hIL-3 + SCF | | | | | |
| Percentage CD15+ | pMON13056 | 57 | 39.6 | 61.1 | 56 | 67.5 |
| Cells at Day 12 of | pMON13056 + SCF | 70.8 | 45.4 | 72.7 | 46.3 | 87.4 |
| CD34+ Cultures | pMON13148 | 47.3 | 58.6 | ND | ND | ND |
| | pMON13418 + SCF | 38.7 | 31.7 | ND | ND | ND |
| | native | 25.6 | 10.5 | 43.3 | 26 | 18.2 |
| | hIL-3 | | | | | |
| | native | 17.7 | 11.5 | 55 | 12.4 | 24.2 |
| | hIL-3 + SCF | | | | | |
| Percentage CD41+ | pMON13056 | 12.6 | 16.5 | 18.2 | 3.4 | 4.6 |
| Cells at Day 12 of | pMON13056 + SCF | 7.4 | 8.3 | 5.5 | 4.8 | 1.8 |
| CD34+ Cultures | pMON13148 | 6 | 9.1 | ND | ND | ND |
| | pmon13148 + SCF | 14.1 | 8.3 | ND | ND | ND |
| | native | 18.9 | 14.1 | 13.7 | 4.2 | 5.5 |
| | hIL-3 | | | | | |
| | native | 15.3 | 10.7 | 12.9 | 7.4 | 15 |
| | hIL-3 + SCF | | | | | |
| Total Number of | pMON13056 | 20 | 49 | ND | 0.8 | 1 |
| Megakaryoctes | pMON13056 + SCF | 40 | 68 | 8.2 | 3.4 | 0.5 |
| (E+05) In Day 10-12 | pMON13148 | 5.2 | 8.7 | ND | ND | ND |
| Cultures | pMON13148 + SCF | 50 | 52 | ND | ND | ND |
| | native | 6.8 | 2.3 | 5.9 | 0.2 | 0.3 |
| | hIL-3 | | | | | |
| | native | 43 | 26 | 32 | 16 | 7.4 |
| | hIL-3 + SCF | | | | | |
| Colony Forming | pMON13056 | 0.9 | 3.2 | ND | 0.2 | 0.1 |
| Unit Granulocyte | pMON13056 + SCF | 1 | 3 | 0.7 | 1.1 | 0.04 |
| Macrophage (CFU- | pMON13148 | 0.5 | 0.8 | ND | ND | ND |
| GM) Index | pMON13148 + SCF | 1.2 | 3.2 | ND | ND | ND |
| | native | 0.2 | 0.06 | 0.03 | 0.03 | 0.03 |
| | hIL-3 | | | | | |
| | native | 1.9 | 1.1 | 0.3 | 0.6 | 0.3 |
| | hIL-3 + SCF | | | | | |
| Colony Forming | pMON13056 | 1.4 | 5.1 | ND | 0.2 | 0.2 |
| Unit-Index | pMON13056 + SCF | 1.3 | 4.3 | 0.3 | 1.1 | 0.2 |
| | pMON13148 | 0.7 | 1.1 | ND | ND | ND |
| | PMON13148 + SCF | 1.6 | 5.2 | ND | ND | ND |
| | native | 0.2 | 0.1 | 0.03 | 0.03 | 0.04 |
| | hIL-3 | | | | | |
| | native | 2.7 | 1.5 | 0.3 | 0.5 | 0.4 |
| | hIL-3 + SCF | | | | | |

### EXAMPLE 5

Ex Vivo Expansion of CD34+ Cells from Bone Marrow Using pMON13056 vs. Native IL-3+/−G-CSF

Cells were cultured as in Example 4 except CD34+ cells were isolated from normal bone marrow. Native IL-3, IL-3 variant (pMON13288) and G-CSF were used at 50 ng/mL and pMON13056 was used at 100 ng/ml of culture medium. Starting cell number for each treatment was 20×10E4. The total cell expansion is shown in Table 5.

TABLE 5

| Treatment | Donor 1 | Donor 2 |
|---|---|---|
| native IL-3 | 42 × 10E4 | 169 × 10E4 |
| pMON13288 | 114 × 10E4 | 259 × 10E4 |
| G-CSF | 14 × 10E4 | 32 × 10E4 |
| pMON13288 | 194 × 10E4 | 609 × 10E4 |
| and G-CSF | | |
| pMON13056 | 219 × 10E4 | 621 × 10E4 |

Amino acids are shown herein by standard one letter or three letter abbreviations as follows:

| Abbreviated | Designation | Amino Acid |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Further details known to those skilled in the art may be found in T. Maniatis, et al., Molecular Cloning, A Laboratory Manual, Cold Spring Harbor Laboratory (1982) and references cited therein, incorporated herein by reference; and in J. Sambrook, et al., Molecular Cloning, A Laboratory Manual, 2nd edition, Cold Spring Harbor Laboratory (1989) and references cited therein, incorporated herein by reference.

Additional details on the IL-3 variants of the present invention may be found in co-pending U.S. patent application Ser. No. 08/411,795 (WO 94/12638) which is hereby incorporated by reference in its entirety as if written herein.

Additional details on how to make the chimera protein can be found in WO 95/21254, WO 92/04455 and WO 91/02754.

Additional details about the lymphokine and the variants thereof can be found in U.S. Pat. Nos. 4,810,643, and 5,218,092 and E.P. Application 02174004.

All references, patents or applications cited herein are incorporated by reference in their entirety as if written herein.

Various other examples will be apparent to the person skilled in the art after reading the present disclosure without departing from the spirit and scope of the invention. It is intended that all such other examples be included within the scope of the appended claims.

1. Cultured stem cells obtained from a method of ex vivo expansion of stem cells, comprising the steps of:
(a) culturing stem cells with a selected growth medium comprising a chimera protein having the formula selected from the group consisting of:
R₁-L-R₂, R₂-L-R₁, R₁-R₂, R₂-R₁, R₁-L-R₁ and R₁-R₁
wherein R₁ is a human interleukin-3 mutant polypeptide of SEQ ID NO:1
wherein
Xaa at position 17 is Ser, Lys, Gly, Asp, Met, Gln, or Arg;
Xaa at position 18 is Asn, His, Leu, Ile, Phe, Arg, or Gln;
Xaa at position 19 is Met, Phe, Ile, Arg, Gly, Ala, or Cys;
Xaa at position 20 is Ile, Cys, Gln, Glu, Arg, Pro, or Ala;
Xaa at position 21 is Asp, Phe, Lys, Arg, Ala, Gly, Glu, Gln, Asn, Thr, Ser or Val;
Xaa at position 22 is Glu, Trp, Pro, Ser, Ala, His, Asp, Asn, Gln, Leu, Val or Gly;
Xaa at position 23 is Ile, Val, Ala, Leu, Gly, Trp, Lys, Phe, Ser, or Arg;
Xaa at position 24 is Ile, Gly, Val, Arg, Ser, Phe, or Leu;
Xaa at position 25 is Thr, His, Gly, Gln, Arg, Pro, or Ala;
Xaa at position 26 is His, Thr, Phe, Gly, Arg, Ala, or Trp;
Xaa at position 27 is Leu, Gly, Arg, Thr, Ser, or Ala;
Xaa at position 28 is Lys, Arg, Leu, Gln, Gly, Pro, Val or Trp;
Xaa at position 29 is Gln, Asn, Leu, Pro, Arg, or Val;
Xaa at position 30 is Pro, His, Thr, Gly, Asp, Gln, Ser, Leu, or Lys;
Xaa at position 31 is Pro, Asp, Gly, Ala, Arg, Leu, or Gln;
Xaa at position 32 is Leu, Val, Arg, Gln, Asn, Gly, Ala, or Glu;
Xaa at position 33 is Pro, Leu, Gln, Ala, Thr, or Glu;
Xaa at position 34 is Leu, Val, Gly, Ser, Lys, Glu, Gln, Thr, Arg, Ala, Phe, Ile or Met;
Xaa at position 35 is Leu, Ala, Gly, Asn, Pro, Gln, or Val;
Xaa at position 36 is Asp, Leu, or Val;
Xaa at position 37 is Phe, Ser, Pro, Trp, or Ile;
Xaa at position 38 is Asn, or Ala;
Xaa at position 40 is Leu, Trp, or Arg;
Xaa at position 41 is Asn, Cys, Arg, Leu, His, Met, or Pro;
Xaa at position 42 is Gly, Asp, Ser, Cys, Asn, Lys, Thr, Leu, Val, Glu, Phe, Tyr, Ile, Met or Ala;
Xaa at position 43 is Glu, Asn, Tyr, Leu, Phe, Asp, Ala, Cys, Gln, Arg, Thr, Gly or Ser;
Xaa at position 44 is Asp, Ser, Leu, Arg, Lys, Thr, Met, Trp, Glu, Asn, Gln, Ala or Pro;
Xaa at position 45 is Gln, Pro, Phe, Val, Met, Leu, Thr, Lys, Trp, Asp, Asn, Arg, Ser, Ala, Ile, Glu or His;
Xaa at position 46 is Asp, Phe, Ser, Thr, Cys, Glu, Asn, Gln, Lys, His, Ala, Tyr, Ile, Val or Gly;
Xaa at position 47 is Ile, Gly, Val, Ser, Arg, Pro, or His;
Xaa at position 48 is Leu, Ser, Cys, Arg, Ile, His, Phe, Glu, Lys, Thr, Ala, Met, Val or Asn;
Xaa at position 49 is Met, Arg, Ala, Gly, Pro, Asn, His, or Asp;
Xaa at position 50 is Glu, Leu, Thr, Asp, Tyr, Lys, Asn, Ser, Ala, Ile, Val, His, Phe, Met or Gln;
Xaa at position 51 is Asn, Arg, Met, Pro, Ser, Thr, or His;
Xaa at position 52 is Asn, His, Arg, Leu, Gly, Ser, or Thr;
Xaa at position 53 is Leu, Thr, Ala, Gly, Glu, Pro, Lys, Ser, or Met;
Xaa at position 54 is Arg, Asp, Ile, Ser, Val, Thr, Gln, Asn, Lys, His, Ala or Leu;
Xaa at position 55 is Arg, Thr, Val, Ser, Leu, or Gly;
Xaa at position 56 is Pro, Gly, Cys, Ser, Gln, Glu, Arg, His, Thr, Ala, Tyr, Phe, Leu, Val or Lys;
Xaa at position 57 is Asn or Gly;
Xaa at position 58 is Leu, Ser, Asp, Arg, Gln, Val, or Cys;
Xaa at position 59 is Glu, Tyr, His, Leu, Pro, or Arg;
Xaa at position 60 is Ala, Ser, Pro, Tyr, Asn, or Thr;
Xaa at position 61 is Phe, Asn, Glu, Pro, Lys, Arg, or Ser;
Xaa at position 62 is Asn, His, Val, Arg, Pro, Thr, Asp, or Ile;
Xaa at position 63 is Arg, Tyr, Trp, Lys, Ser, His, Pro, or Val;
Xaa at position 64 is Ala, Asn, Pro, Ser, or Lys;
Xaa at position 65 is Val, Thr, Pro, His, Leu, Phe, or Ser;
Xaa at position 66 is Lys, Ile, Arg, Val, Asn, Glu, or Ser;
Xaa at position 67 is Ser, Ala, Phe, Val, Gly, Asn, Ile, Pro, or His;
Xaa at position 68 is Leu, Val, Trp, Ser, Ile, Phe, Thr, or His;
Xaa at position 69 is Gln, Ala, Pro, Thr, Glu, Arg, Trp, Gly, or Leu;
Xaa at position 70 is Asn, Leu, Val, Trp, Pro, or Ala;
Xaa at position 71 is Ala, Met, Leu, Pro, Arg, Glu, Thr, Gln, Trp, or Asn;
Xaa at position 72 is Ser, Glu, Met, Ala, His, Asn, Arg, or Asp;
Xaa at position 73 is Ala, Glu, Asp, Leu, Ser, Gly, Thr, or Arg;
Xaa at position 74 is Ile, Met, Thr, Pro, Arg, Gly, Ala;
Xaa at position 75 is Glu, Lys, Gly, Asp, Pro, Trp, Arg, Ser, Gln, or Leu;
Xaa at position 76 is Ser, Val, Ala, Asn, Trp, Glu, Pro, Gly, or Asp;
Xaa at position 77 is Ile, Ser, Arg, Thr, or Leu;
Xaa at position 78 is Leu, Ala, Ser, Glu, Phe, Gly, or Arg;
Xaa at position 79 is Lys, Thr, Asn, Met, Arg, Ile, Gly, or Asp;
Xaa at position 80 is Asn, Trp, Val, Gly, Thr, Leu, Glu, or Arg;
Xaa at position 81 is Leu, Gln, Gly, Ala, Trp, Arg, Val, or Lys;
Xaa at position 82 is Leu, Gln, Lys, Trp, Arg, Asp, Glu, Asn, His, Thr, Ser, Ala, Tyr, Phe, Ile, Met or Val;
Xaa at position 83 is Pro, Ala, Thr, Trp, Arg, or Met;
Xaa at position 84 is Cys, Glu, Gly, Arg, Met, or Val;
Xaa at position 85 is Leu, Asn, Val, or Gln;
Xaa at position 86 is Pro, Cys, Arg, Ala, or Lys;
Xaa at position 87 is Leu, Ser, Trp, or Gly;
Xaa at position 88 is Ala, Lys, Arg, Val, or Trp;
Xaa at position 89 is Thr, Asp, Cys, Leu, Val, Glu, His, Asn, or Ser;
Xaa at position 90 is Ala, Pro, Ser, Thr, Gly, Asp, Ile, or Met;
Xaa at position 91 is Ala, Pro, Ser, Thr, Phe, Leu, Asp, or His;
Xaa at position 92 is Pro, Phe, Arg, Ser, Lys, His, Ala, Gly, Ile or Leu;
Xaa at position 93 is Thr, Asp, Ser, Asn, Pro, Ala, Leu, or Arg;
Xaa at position 94 is Arg, Ile, Ser, Glu, Leu, Val, Gln, Lys, His, Ala, or Pro;
Xaa at position 95 is His, Gln, Pro, Arg, Val, Leu, Gly, Thr, Asn, Lys, Ser, Ala, Trp, Phe, Ile, or Tyr;
Xaa at position 96 is Pro, Lys, Tyr, Gly, Ile, or Thr;
Xaa at position 97 is Ile, Val, Lys, Ala, or Asn;
Xaa at position 98 is His, Ile, Asn, Leu, Asp, Ala, Thr, Glu, Gln, Ser, Phe, Met, Val, Lys, Arg, Tyr or Pro;
Xaa at position 99 is Ile, Leu, Arg, Asp, Val, Pro, Gln, Gly, Ser, Phe, or His;
Xaa at position 100 is Lys, Tyr, Leu, His, Arg, Ile, Ser, Gln, or Pro;
Xaa at position 101 is Asp, Pro, Met, Lys, His, Thr, Val, Tyr, Glu, Asn, Ser, Ala, Gly, Ile, Leu, or Gln;
Xaa at position 102 is Gly, Leu, Glu, Lys, Ser, Tyr, or Pro;
Xaa at position 103 is Asp, or Ser;
Xaa at position 104 is Trp, Val, Cys, Tyr, Thr, Met, Pro, Leu, Gln, Lys, Ala, Phe, or Gly;
Xaa at position 105 is Asn, Pro, Ala, Phe, Ser, Trp, Gln, Tyr, Leu, Lys, Ile, Asp, or His;
Xaa at position 106 is Glu, Ser, Ala, Lys, Thr, Ile, Gly, or Pro;
Xaa at position 108 is Arg, Lys, Asp, Leu, Thr, Ile, Gln, His, Ser, Ala or Pro;
Xaa at position 109 is Arg, Thr, Pro, Glu, Tyr, Leu, Ser, or Gly;
Xaa at position 110 is Lys, Ala, Asn, Thr, Leu, Arg, Gln, His, Glu, Ser, or Trp;
Xaa at position 111 is Leu, Ile, Arg, Asp, or Met;
Xaa at position 112 is Thr, Val, Gln, Tyr, Glu, His, Ser, or Phe;
Xaa at position 113 is Phe, Ser, Cys, His, Gly, Trp, Tyr, Asp, Lys, Leu, Ile, Val or Asn;
Xaa at position 114 is Tyr, Cys, His, Ser, Trp, Arg, or Leu;
Xaa at position 115 is Leu, Asn, Val, Pro, Arg, Ala, His, Thr, Trp, or Met;
Xaa at position 116 is Lys, Leu, Pro, Thr, Met, Asp, Val, Glu, Arg, Trp, Ser, Asn, His, Ala, Tyr, Phe, Gln, or Ile;
Xaa at position 117 is Thr, Ser, Asn, Ile, Trp, Lys, or Pro;
Xaa at position 118 is Leu, Ser, Pro, Ala, Glu, Cys, Asp, or Tyr;
Xaa at position 119 is Glu, Ser, Lys, Pro, Leu, Thr, Tyr, or Arg;
Xaa at position 120 is Asn, Ala, Pro, Leu, His, Val, or Gln;
Xaa at position 121 is Ala, Ser, Ile, Asn, Pro, Lys, Asp, or Gly;
Xaa at position 122 is Gln, Ser, Met, Trp, Arg, Phe, Pro, His, Ile, Tyr, or Cys;
Xaa at position 123 is Ala, Met, Glu, His, Ser, Pro, Tyr, or Leu;
wherein from 1 to 14 amino acids can be deleted from the N-terminus and/or from 1 to 15 amino acids can be deleted from the C-terminus of said human interleukin-3 mutant polypeptide;
and wherein from 4 to 44 of the amino acids designated by Xaa are different from the corresponding amino acids of native (1-133) human interleukin-3;
R₂ is a hematopoietic growth factor;
L is a linker capable of Linking R₁ to R₂; and said chimera protein can additionally be immediately preceded by (methionine ⁻¹), (alanine⁻¹), or (methionine⁻², alanine⁻¹), and
(b) harvesting said cultured stem cells.

2. The cultured stem cells of claim 1 wherein said chimera protein is of the formula selected from the group consisting of:
R₁-L-R₂, R₂-L-R₁, R₁-R₂, R₂-R₁, R₁-L-R₁ and R₁-R₁
wherein R₁ is a human interleukin-3 mutant polypeptide of SEQ ID NO:4
wherein
Xaa at position 3 is Ser, Lys, Gly, Asp, Met, Gln, or Arg;
Xaa at position 4 is Asn, His, Leu, Ile, Phe, Arg, or Gln;
Xaa at position 5 is Met, Phe, Ile, Arg, Gly, Ala, or Cys;
Xaa at position 6 is Ile, Cys, Gln, Glu, Arg, Pro, or Ala;
Xaa at position 7 is Asp, Phe, Lys, Arg, Ala, Gly, Glu, Gln, Asn, Thr, Ser or Val;
Xaa at position 8 is Glu, Trp, Pro, Ser, Ala, His, Asp, Asn, Gln, Leu, Val, or Gly;
Xaa at position 9 is Ile, Val, Ala, Gly, Trp, Lys, Phe, Ser, or Arg;
Xaa at position 10 is Ile, Gly, Val, Arg, Ser, Phe, or Leu;
Xaa at position 11 is Thr, His, Gly, Gln, Arg, Pro, or Ala;
Xaa at position 12 is His, Thr, Phe, Gly, Arg, Ala, or Trp;
Xaa at position 13 is Leu, Gly, Arg, Thr, Ser, or Ala;
Xaa at position 14 is Lys, Arg, Leu, Gln, Gly, Pro, Val or Trp;
Xaa at position 15 is Gln, Asn, Leu, Pro, Arg, or Val;
Xaa at position 16 is Pro, His, Thr, Gly, Asp, Gln, Ser, Leu, or Lys;
Xaa at position 17 is Pro, Asp, Gly, Ala, Arg, Leu, or Gln;
Xaa at position 18 is Leu, Val, Arg, Gln, Asn, Gly, Ala, or Glu;
Xaa at position 19 is Pro, Leu, Gln, Ala, Thr, or Glu;
Xaa at position 20 is Leu, Val, Gly, Ser, Lys, Glu, Gln, Thr, Arg, Ala, Phe, Ile or Met;
Xaa at position 21 is Leu, Ala, Gly, Asn, Pro, Gln, or Val;
Xaa at position 22 is Asp, Leu, or Val;
Xaa at position 23 is Phe, Ser, Pro, Trp, or Ile;
Xaa at position 24 is Asn, or Ala;
Xaa at position 26 is Leu, Trp, or Arg;
Xaa at position 27 is Asn, Cys, Arg, Leu, His, Met, Pro;
Xaa at position 28 is Gly, Asp, Ser, Cys, Ala, Lys, Asn, Thr, Leu, Val, Glu, Phe, Tyr, Ile or Met;
Xaa at position 29 is Glu, Asn, Tyr, Leu, Phe, Asp, Ala, Cys, Gln, Arg, Thr, Gly or Ser;
Xaa at position 30 is Asp, Ser, Leu, Arg, Lys, Thr, Met, Trp, Glu, Asn, Gln, Ala or Pro;
Xaa at position 31 is Gln, Pro, Phe, Val, Met, Leu, Thr, Lys, Asp, Asn, Arg, Ser, Ala, Ile, Glu, His or Trp;
Xaa at position 32 is Asp, Phe, Ser, Thr, Cys, Glu, Asn, Gln, Lys, His, Ala, Tyr, Ile, Val or Gly;
Xaa at position 33 is Ile, Gly, Val, Ser, Arg, Pro, or His;
Xaa at position 34 is Leu, Ser, Cys, Arg, Ile, His, Phe, Glu, Lys, Thr, Ala, Met, Val or Asn;
Xaa at position 35 is Met, Arg, Ala, Gly, Pro, Asn, His, or Asp;
Xaa at position 36 is Glu, Leu, Thr, Asp, Tyr, Lys, Asn, Ser, Ala, Ile, Val, His, Phe, Met or Gln;
Xaa at position 37 is Asn, Arg, Met, Pro, Ser, Thr, or His;
Xaa at position 38 is Asn, His, Arg, Leu, Gly, Ser, or Thr;
Xaa at position 39 is Leu, Thr, Ala, Gly, Glu, Pro, Lys, Ser, or Met;
Xaa at position 40 is Arg, Asp, Ile, Ser, Val, Thr, Gln, Asn, Lys, His, Ala or Leu;
Xaa at position 41 is Arg, Thr, Val, Ser, Leu, or Gly;
Xaa at position 42 is Pro, Gly, Cys, Ser, Gln, Glu, Arg, His, Thr, Ala, Tyr, Phe, Leu, Val or Lys;
Xaa at position 43 is Asn or Gly;
Xaa at position 44 is Leu, Ser, Asp, Arg, Gln, Val, or Cys;
Xaa at position 45 is Glu, Tyr, His, Leu, Pro, or Arg;
Xaa at position 46 is Ala, Ser, Pro, Tyr, Asn, or Thr;
Xaa at position 47 is Phe, Asn, Glu, Pro, Lys, Arg, or Ser;
Xaa at position 48 is Asn, His, Val, Arg, Pro, Thr, Asp, or Ile;
Xaa at position 49 is Arg, Tyr, Trp, Lys, Ser, His, Pro, or Val;
Xaa at position 50 is Ala, Asn, Pro, Ser, or Lys;
Xaa at position 51 is Val, Thr, Pro, His, Leu, Phe, or Ser;
Xaa at position 52 is Lys, Ile, Arg, Val, Asn, Glu, or Ser;
Xaa at position 53 is Ser, Ala, Phe, Val, Gly, Asn, Ile, Pro, or His;
Xaa at position 54 is Leu, Val, Trp, Ser, Ile, Phe, Thr, or His;
Xaa at position 55 is Gln, Ala, Pro, Thr, Glu, Arg, Trp, Gly, or Leu;
Xaa at position 56 is Asn, Leu, Val, Trp, Pro, or Ala;
Xaa at position 57 is Ala, Met, Leu, Pro, Arg, Glu, Thr, Gln, Trp, or Asn;
Xaa at position 58 is Ser, Glu, Met, Ala, His, Asn, Arg, or Asp;
Xaa at position 59 is Ala, Glu, Asp, Leu, Ser, Gly, Thr, or Arg;
Xaa at position 60 is Ile, Met, Thr, Pro, Arg, Gly, Ala;
Xaa at position 61 is Glu, Lys, Gly, Asp, Pro, Trp, Arg, Ser, Gln, or Leu;
Xaa at position 62 is Ser, Val, Ala, Asn, Trp, Glu, Pro, Gly, or Asp;
Xaa at position 63 is Ile, Ser, Arg, Thr, or Leu;
Xaa at position 64 is Leu, Ala, Ser, Glu, Phe, Gly, or Arg;
Xaa at position 65 is Lys, Thr, Gly, Asn, Met, Arg, Ile, or Asp;
Xaa at position 66 is Asn, Trp, Val, Gly, Thr, Leu, Glu, or Arg;
Xaa at position 67 is Leu, Gln, Gly, Ala, Trp, Arg, Val, or Lys;
Xaa at position 68 is Leu, Gln, Lys, Trp, Arg, Asp, Glu, Asn, His, Thr, Ser, Ala, Tyr, Phe, Ile, Met or Val;
Xaa at position 69 is Pro, Ala, Thr, Trp, Arg, or Met;
Xaa at position 70 is Cys, Glu, Gly, Arg, Met, or Val;
Xaa at position 71 is Leu, Asn, Val, or Gln;
Xaa at position 72 is Pro, Cys, Arg, Ala, or Lys;
Xaa at position 73 is Leu, Ser, Trp, or Gly;
Xaa at position 74 is Ala, Lys, Arg, Val, or Trp;
Xaa at position 75 is Thr, Asp, Cys, Leu, Val, Glu, His, Asn, or Ser;
Xaa at position 76 is Ala, Pro, Ser, Thr, Gly, Asp, Ile, or Met;
Xaa at position 77 is Ala, Pro, Ser, Thr, Phe, Leu, Asp, or His;
Xaa at position 78 is Pro, Phe, Arg, Ser, Lys, His, Ala, Gly, Ile or Leu;
Xaa at position 79 is Thr, Asp, Ser, Asn, Pro, Ala, Leu, or Arg;
Xaa at position 80 is Arg, Ile, Ser, Glu, Leu, Val, Gln, Lys, His, Ala or Pro;
Xaa at position 81 is His, Gln, Pro, Arg, Val, Leu, Gly, Thr, Asn, Lys, Ser, Ala, Trp, Phe, Ile or Tyr;
Xaa at position 82 is Pro, Lys, Tyr, Gly, Ile, or Thr;
Xaa at position 83 is Ile, Val, Lys, Ala, or Asn;
Xaa at position 84 is His, Ile, Asn, Leu, Asp, Ala, Thr, Glu, Gln, Ser, Phe, Met, Val, Lys, Arg, Tyr or Pro;
Xaa at position 85 is Ile, Leu, Arg, Asp, Val, Pro, Gln, Gly, Ser, Phe, or His;
Xaa at position 86 is Lys, Tyr, Leu, His, Arg, Ile, Ser, Gln, or Pro;
Xaa at position 87 is Asp, Pro, Met, Lys, His, Thr, Val, Tyr, Glu, Asn, Ser, Ala, Gly, Ile, Leu or Gln;
Xaa at position 88 is Gly, Leu, Glu, Lys, Ser, Tyr, or Pro;
Xaa at position 89 is Asp, or Ser;
Xaa at position 90 is Trp, Val, Cys, Tyr, Thr, Met, Pro, Leu, Gln, Lys, Ala, Phe, or Gly;
Xaa at position 91 is Asn, Pro, Ala, Phe, Ser, Trp, Gln, Tyr, Leu, Lys, Ile, Asp, or His;
Xaa at position 92 is Glu, Ser, Ala, Lys, Thr, Ile, Gly, or Pro;
Xaa at position 94 is Arg, Lys, Asp, Leu, Thr, Ile, Gln, His, Ser, Ala, or Pro;
Xaa at position 95 is Arg, Thr, Pro, Glu, Tyr, Leu, Ser, or Gly;
Xaa at position 96 is Lys, Asn, Thr, Leu, Gln, Arg, His, Glu, Ser, Ala or Trp;
Xaa at position 97 is Leu, Ile, Arg, Asp, or Met;
Xaa at position 98 is Thr, Val, Gln, Tyr, Glu, His, Ser, or Phe;
Xaa at position 99 is Phe, Ser, Cys, His, Gly, Trp, Tyr, Asp, Lys, Leu, Ile, Val or Asn;
Xaa at position 100 is Tyr, Cys, His, Ser, Trp, Arg, or Leu;
Xaa at position 101 is Leu, Asn, Val, Pro, Arg, Ala, His, Thr, Trp, or Met;
Xaa at position 102 is Lys, Leu, Pro, Thr, Met, Asp, Val, Glu, Arg, Trp, Ser, Asn, His, Ala, Tyr, Phe, Gln, or Ile;
Xaa at position 103 is Thr, Ser, Asn, Ile, Trp, Lys, or Pro;
Xaa at position 104 is Leu, Ser, Pro, Ala, Glu, Cys, Asp, or Tyr;
Xaa at position 105 is Glu, Ser, Lys, Pro, Leu, Thr, Tyr, or Arg;
Xaa at position 106 is Asn, Ala, Pro, Leu, His, Val, or Gln;
Xaa at position 107 is Ala, Ser, Ile, Asn, Pro, Lys, Asp, or Gly;
Xaa at position 108 is Gln, Ser, Met, Trp, Arg, Phe, Pro, His, Ile, Tyr, or Cys;
Xaa at position 109 is Ala, Met, Glu, His, Ser, Pro, Tyr, or Leu;
wherein from 4 to 44 of the amino acids designated by Xaa are different from the corresponding native amino acids of (1-133) human interleukin-3;
R₂ is a hematopoietic growth factor;
L is a linker capable of Linking R₁ to R₂; and said chimera protein can additionally be immediately preceded by (methionine⁻¹), (alanine⁻¹), or (methionine⁻², alanine⁻¹).

3. Cultured stem cells obtained from a method of ex vivo expansion of stem cells, comprising the steps of:
(a) culturing stem cells with a selected growth medium comprising a chimera protein having the formula selected from the group consisting of:
R₁-L-R₂, R₂-L-R₁, R₁-R₂, R₂-R₁, R₁-L-R₁ and R₁-R₁
wherein R₁ is a human interleukin-3 mutant polypeptide of SEQ ID NO:7
wherein m is 0 or 1; Xaa at position 18 is Asn or Ile; Xaa at position 19 is Met, Ala or Ile; Xaa at position 20 is Ile, Pro or Leu; Xaa at position 23 is Ile, Ala or Leu; Xaa at position 25 is Thr or His; Xaa at position 29 is Gln, Arg, Val or Leu; Xaa at position 32 is Leu, Ala, Asn or Arg; Xaa at position 34 is Leu or Ser; Xaa at position 37 is Phe, Pro, or Ser; Xaa at position 38 is Asn or Ala; Xaa at position 42 is Gly, Ala, Ser, Asp or Asn; Xaa at position 45 is Gln, Val, or Met; Xaa at position 46 is Asp or Ser; Xaa at position 49 is Met, Ile, Leu or Asp; Xaa at position 50 is Glu or Asp; Xaa at position 51 is Asn Arg or Ser; Xaa at position 55 is Arg, Leu, or Thr; Xaa at position 56 is Pro or Ser; Xaa at position 59 is Glu or Leu; Xaa at position 60 is Ala or Ser; Xaa at position 62 is Asn, Val or Pro; Xaa at position 63 is Arg or His; Xaa at position 65 is Val or Ser; Xaa at position 67 is Ser, Asn, His or Gly; Xaa at position 69 is Gln or Glu; Xaa at position 73 is Ala or Gly; Xaa at position 76 is Ser, Ala or Pro; Xaa at position 79 is Lys, Arg or Ser; Xaa at position 82 is Leu, Glu, Val or Trp; Xaa at position 85 is Leu or Val; Xaa at position 87 is Leu, Ser, Trp; Xaa at position 88 is Ala or Trp; Xaa at position 91 is Ala or Pro; Xaa at position 93 is Pro or Ser; Xaa at position 95 is His or Thr; Xaa at position 98 is His, Ile, or Thr; Xaa at position 100 is Lys or Arg; Xaa at position 101 is Asp, Ala or Met; Xaa at position 105 is Asn or Gln; Xaa at position 109 is Arg, Glu or Leu; Xaa at position 112 is Thr or Gln; Xaa at position 116 is Lys, Val, Trp or Ser; Xaa at position 117 is Thr or Ser; Xaa at position 120 is Asn, Gln, or His; Xaa at position 123 is Ala or Glu; with the proviso that from four to forty-four of the amino acids designated by Xaa are different from the corresponding amino acids of native human interleukin-3);
R₂ is a hematopoietic growth factor;
L is a linker capable of Linking R₁ to R₂; and said chimera protein can additionally be immediately preceded by (methionine⁻¹), (alanine⁻¹), or (methionine⁻², alanine⁻¹); and
(b) harvesting said cultured stem cells.

4. Cultured stem cells obtained by the method of ex vivo expansion of stem cells, comprising the steps of:
(a) culturing said stem cells with a selected growth medium comprising a chimera protein having the formula selected from the group consisting of:
R₁-L-R₂, R₂-L-R₁, R₁-R₂, R₂-R₁, R₁-L-R₁ and R₁-R₁
wherein R₁ is a human interleukin-3 mutant polypeptide of SEQ ID NO:8
wherein m is 0 or 1; n is 0 or 1; p is 0 or 1; Xaa at position 4 is Asn or Ile; Xaa at position 5 is Met, Ala or Ile: Xaa at position 6 is Ile, Pro or Leu; Xaa at position 9 is Ile, Ala or Leu; Xaa at position 11 is Thr or His; Xaa at position 15 is Gln, Arg, Val or Leu; Xaa at position 18 is Leu, Ala, Asn or Arg; Xaa at position 20 is Leu or Ser; Xaa at position 23 is Phe, Pro, or Ser; Xaa at position 24 is Asn or Ala; Xaa at position 28 is Gly, Ala, Ser, Asp or Asn; Xaa at position 31 is Gln, Val, or Met; Xaa at position 32 is Asp or Ser; Xaa at position 35 is Met, Ile, Leu or Asp; Xaa at position 36 is Glu or Asp; Xaa at position 37 is Asn, Arg or Ser; Xaa at position 41 is Arg, Leu, or Thr; Xaa at position 42 is Pro or Ser; Xaa at position 45 is Glu or Leu; Xaa at position 46 is Ala or Ser; Xaa at position 48 is Asn, Val or Pro; Xaa at position 49 is Arg or His; Xaa at position 51 is Val or Ser; Xaa at position 53 is Ser, Asn, His or Gly; Xaa at position 55 is Gln or Glu; Xaa at position 59 is Ala or Gly; Xaa at position 62 is Ser, Ala or Pro; Xaa at position 65 is Lys, Arg or Ser; Xaa at position 67 is Leu, Glu, or Val; Xaa at position 68 is Leu, Glu, Val or Trp; Xaa at position 71 is Leu or Val; Xaa at position 73 is Leu, Ser or Trp; Xaa at position 74 is Ala or Trp; Xaa at position 77 is Ala or Pro; Xaa at position 79 is Pro or Ser; Xaa at position 81 is His or Thr; Xaa at position 84 is His, Ile, or Thr; Xaa at position 86 is Lys or Arg; Xaa at position 87 is Asp, Ala or Met; Xaa at position 91 is Asn or Glu; Xaa at position 95 is Arg, Glu, Leu; Xaa at position 98 Thr or Gln; Xaa at position 102 is Lys, Val, Trp or Ser; Xaa at position 103 is Thr or Ser; Xaa at position 106 is Asn, Gln, or His; Xaa at position 109 is Ala or Glu; with the proviso that from four to forty-four of the amino acids designated by Xaa are different from the corresponding amino acids of native (15-125)human interleukin-3;
R₂ is a hematopoietic growth factor;
L is a linker capable of Linking R₁ to R₂; and said chimera protein can additionally be immediately preceded by (methionine⁻¹), (alanine⁻¹), or (methionine⁻², alanine⁻¹); and
(b) harvesting said cultured stem cells.

5. The cultured stem cells of claim 1 wherein R₁ is selected from the group consisting of:

| | |
|---|---|
| Asn Cys Ser Ile Met Ile Asp Glu Ile | SEQ ID NO:9 |
| Ile His His Leu Lys Arg Pro Pro Ala | |
| Pro Leu Leu Asp Pro Asn Asn Leu Asn | |
| Ala Glu Asp Val Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Ile Met Ile Asp Glu Ile | SEQ ID NO:10 |
| Ile His His Leu Lys Arg Pro Pro Asn | |
| Pro Leu Leu Asp Pro Asn Asn Leu Asn | |
| Ser Glu Asp Met Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Ile Met Ile Asp Glu Ile | SEQ ID NO:11 |
| Ile His His Leu Lys Val Pro Pro Ala | |
| Pro Leu Leu Asp Ser Asn Asn Leu Asn | |
| Ser Glu Asp Met Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:12 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Arg Asn Leu Arg Leu Pro Asn Leu Leu | |
| Ala Phe Val Arg Ala Val Lys Asn Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:13 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Arg Asn Leu Arg Leu Pro Asn Leu Glu | |
| Ser Phe Val Arg Ala Val Lys Asn Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:14 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Arg Asn Leu Arg Thr Pro Asn Leu Leu | |
| Ala Phe Val Arg Ala Val Lys His Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:15 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Gly Ile Glu Ala Ile | |
| Leu Arg Asn Leu Gln Pro Cys Leu Pro | |
| Ser Ala Thr Ala Ala Pro Ser Arg His | |
| Pro Ile Ile Ile Lys Ala Gly Asp Trp | |
| Gln Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:16 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Gly Ile Glu Ala Ile | |
| Leu Arg Asn Leu Val Pro Cys Leu Pro | |
| Ser Ala Thr Ala Ala Pro Ser Arg His | |
| Pro Ile Thr Ile Lys Ala Gly Asp Trp | |
| Gln Glu Phe Arg Arg Lys Leu Thr Phe | |
| Tyr Leu Lys Thr Leu Glu Asn Ala Gln | |
| Ala Gln Gln; | |
| Asn Cys Ser Asn Met Ile Asp Glu Ile | SEQ ID NO:17 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Glu Lys Leu Thr Phe | |
| Tyr Leu Val Thr Leu Glu Gln Ala Gln | |
| Glu Gln Gln; | |
| Asn Cys Ser Asn Met Ile Asp Glu Ile | SEQ ID NO:18 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Glu Lys Leu Thr Phe | |
| Tyr Leu Val Ser Leu Glu His Ala Gln | |
| Glu Gln Gln; | |
| Asn Cys Ser Asn Met Ile Asp Glu Ile | SEQ ID NO:19 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Gly Ile Glu Ala Ile | |
| Leu Arg Asn Leu Gln Pro Cys Leu Pro | |
| Ser Ala Thr Ala Ala Pro Ser Arg His | |
| Pro Ile Ile Ile Lys Ala Gly Asp Trp | |
| Gln Glu Phe Arg Glu Lys Leu Thr Phe | |
| Tyr Leu Val Thr Leu Glu Gln Ala Gln | |
| Glu Gln Gln; | |
| Asn Cys Ser Asn Met Ile Asp Glu Ile | SEQ ID NO:20 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Gly Ile Glu Ala Ile | |
| Leu Arg Asn Leu Val Pro Cys Leu Pro | |
| Ser Ala Thr Ala Ala Pro Ser Arg His | |
| Pro Ile Thr Ile Lys Ala Gly Asp Trp | |
| Gln Glu Phe Arg Glu Lys Leu Thr Phe | |
| Tyr Leu Val Thr Leu Glu Gln Ala Gln | |
| Glu Gln Gln; | |
| Asn Cys Ser Asn Met Ile Asp Glu Ile | SEQ ID NO:21 |
| Ile Thr His Leu Lys Gln Pro Pro Leu | |
| Pro Leu Leu Asp Phe Asn Asn Leu Asn | |
| Gly Glu Asp Gln Asp Ile Leu Met Glu | |
| Asn Asn Leu Arg Arg Pro Asn Leu Glu | |
| Ala Phe Asn Arg Ala Val Lys Ser Leu | |
| Gln Asn Ala Ser Gly Ile Glu Ala Ile | |
| Leu Arg Asn Leu Val Pro Cys Leu Pro | |
| Ser Ala Thr Ala Ala Pro Ser Arg His | |
| Pro Ile Thr Ile Lys Ala Gly Asp Trp | |
| Gln Glu Phe Arg Glu Lys Leu Thr Phe | |
| Tyr Leu Val Ser Leu Glu His Ala Gln | |
| Glu Gln Gln; | |
| Asn Cys Ser Ile Met Ile Asp Glu Ile | SEQ ID NO:22 |
| Ile His His Leu Lys Arg Pro Pro Ala | |
| Pro Leu Leu Asp Pro Asn Asn Leu Asn | |
| Ala Glu Asp Val Asp Ile Leu Met Glu | |
| Arg Asn Leu Arg Leu Pro Asn Leu Glu | |
| Ser Phe Val Arg Ala Val Lys Asn Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Ile Met Ile Asp Glu Ile | SEQ ID NO:23 |
| Ile His His Leu Lys Arg Pro Pro Asn | |
| Pro Leu Leu Asp Pro Asn Asn Leu Asn | |
| Ser Glu Asp Met Asp Ile Leu Met Glu | |
| Arg Asn Leu Arg Thr Pro Asn Leu Leu | |
| Ala Phe Val Arg Ala Val Lys His Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Asn Cys Ser Ile Met Ile Asp Glu Ile | SEQ ID NO:24 |
| Ile His His Leu Lys Val Pro Pro Ala | |
| Pro Leu Leu Asp Ser Asn Asn Leu Asn | |
| Ser Glu Asp Met Asp Ile Leu Met Glu | |
| Arg Asn Leu Arg Leu Pro Asn Leu Leu | |
| Ala Phe Val Arg Ala Val Lys Asn Leu | |
| Glu Asn Ala Ser Ala Ile Glu Ser Ile | |
| Leu Lys Asn Leu Leu Pro Cys Leu Pro | |
| Leu Ala Thr Ala Ala Pro Thr Arg His | |
| Pro Ile His Ile Lys Asp Gly Asp Trp | |
| Asn Glu Phe Arg Arg Lys Leu Thr Phe | |
| Tyr Leu Lys Thr Leu Glu Asn Ala Gln | |
| Ala Gln Gln; | |
| Met Ala Asn Cys Ser Asn Met Ile Asp | SEQ ID NO:25 |
| Glu Ile Ile Thr His Leu Lys Gln Pro | |
| Pro Leu Pro Leu Leu Asp Phe Asn Asn | |
| Leu Asn Gly Glu Asp Gln Asp Ile Leu | |
| Met Glu Asn Asn Leu Arg Arg Pro Asn | |
| Leu Glu Ala Phe Asn Arg Ala Val Lys | |
| Ser Leu Gln Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Asn Met Ile Asp | SEQ ID NO:26 |
| Glu Ile Ile Thr His Leu Lys Gln Pro | |
| Pro Leu Pro Leu Leu Asp Phe Asn Asn | |
| Leu Asn Gly Glu Asp Gln Asp Ile Leu | |
| Met Glu Asn Asn Leu Arg Arg Pro Asn | |
| Leu Glu Ala Phe Asn Arg Ala Val Lys | |
| Ser Leu Gln Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Asn Met Ile Asp | SEQ ID NO:27 |
| Glu Ile Ile Thr His Leu Lys Gln Pro | |
| Pro Leu Pro Leu Leu Asp Phe Asn Asn | |
| Leu Asn Gly Glu Asp Gln Asp Ile Leu | |
| Met Glu Asn Asn Leu Arg Arg Pro Asn | |
| Leu Glu Ala Phe Asn Arg Ala Val Lys | |
| Ser Leu Gln Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Ser Leu Glu His | |
| Ala Gln Glu Gln Gln; | |
| Met Ala Asn Cys Ser Ile Met Ile Asp | SEQ ID NO:28 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Glu Arg Asn Leu Arg Leu Pro Asn | |
| Leu Glu Ser Phe Val Arg Ala Val Lys | |
| Asn Leu Glu Asn Ala Ser Ala Ile Glu | |
| Ser Ile Leu Lys Asn Leu Leu Pro Cys | |
| Leu Pro Leu Ala Thr Ala Ala Pro Thr | |
| Arg His Pro Ile His Ile Lys Asp Gly | |
| Asp Trp Asn Glu Phe Arg Arg Lys Leu | |
| Thr Phe Tyr Leu Lys Thr Leu Glu Asn | |
| Ala Gln Ala Gln Gln; | |
| Met Ala Asn Cys Ser Ile Met Ile Asp | SEQ ID NO:29 |
| Glu Ile Ile His His Leu Lys Arg Pro | |
| Pro Asn Pro Leu Leu Asp Pro Asn Asn | |
| Leu Asn Ser Glu Asp Met Asp Ile Leu | |
| Met Glu Arg Asn Leu Arg Thr Pro Asn | |
| Leu Leu Ala Phe Val Arg Ala Val Lys | |
| His Leu Glu Asn Ala Ser Ala Ile Glu | |
| Ser Ile Leu Lys Asn Leu Leu Pro Cys | |
| Leu Pro Leu Ala Thr Ala Ala Pro Thr | |
| Arg His Pro Ile His Ile Lys Asp Gly | |
| Asp Trp Asn Glu Phe Arg Arg Lys Leu | |
| Thr Phe Tyr Leu Lys Thr Leu Glu Asn | |
| Ala Gln Ala Gln Gln; | |
| Met Ala Asn Cys Ser Ile Met Ile Asp | SEQ ID NO:30 |
| Glu Ile Ile His His Leu Lys Val Pro | |
| Pro Ala Pro Leu Leu Asp Ser Asn Asn | |
| Leu Asn Ser Glu Asp Met Asp Ile Leu | |
| Met Glu Arg Asn Leu Arg Leu Pro Asn | |
| Leu Leu Ala Phe Val Arg Ala Val Lys | |
| Asn Leu Glu Asn Ala Ser Ala Ile Glu | |
| Ser Ile Leu Lys Asn Leu Leu Pro Cys | |
| Leu Pro Leu Ala Thr Ala Ala Pro Thr | |
| Arg His Pro Ile His Ile Lys Asp Gly | |
| Asp Trp Asn Glu Phe Arg Arg Lys Leu | |
| Thr Phe Tyr Leu Lys Thr Leu Glu Asn | |
| Ala Gln Ala Gln Gln; | |
| Met Ala Asn Cys Ser Ile Met Ile Asp | SEQ ID NO:31 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:32 |
| Glu Ile Ile His His Leu Lys Arg Pro | |
| Pro Asn Pro Leu Leu Asp Pro Asn Asn | |
| Leu Asn Ser Glu Asp Met Asp Ile Leu | |
| Met Glu Arg Asn Leu Arg Thr Pro Asn | |
| Leu Leu Ala Phe Val Arg Ala Val Lys | |
| His Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:33 |
| Glu Ile Ile His His Leu Lys Val Pro | |
| Pro Ala Pro Leu Leu Asp Ser Asn Asn | |
| Leu Asn Ser Glu Asp Met Asp Ile Leu | |
| Met Glu Arg Asn Leu Arg Leu Pro Asn | |
| Leu Leu Ala Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:34 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:35 |
| Glu Ile Ile His His Leu Lys Val Pro | |
| Pro Ala Pro Leu Leu Asp Ser Asn Asn | |
| Leu Asn Ser Glu Asp Met Asp Ile Leu | |
| Met Glu Arg Asn Leu Arg Leu Pro Asn | |
| Leu Leu Ala Phe Val Arg Ala Val Lys | |
| Asn Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:36 |
| Glu Ile Ile His His Leu Lys Arg Pro | |
| Pro Asn Pro Leu Leu Asp Pro Asn Asn | |
| Leu Asn Ser Glu Asp Met Asp Ile Leu | |
| Met Glu Arg Asn Leu Arg Thr Pro Asn | |
| Leu Leu Ala Phe Val Arg Ala Val Lys | |
| His Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Ser Leu Glu His | |
| Ala Gln Glu Gln Gln; | |
| Met Ala Asn Cys Ser Ile Met Ile Asp | SEQ ID NO:37 |
| Glu Ile Ile His His Leu Lys Val Pro | |
| Pro Ala Pro Leu Leu Asp Ser Asn Asn | |
| Leu Asn Ser Glu Asp Met Asp Ile Leu | |
| Met Glu Arg Asn Leu Arg Leu Pro Asn | |
| Leu Leu Ala Phe Val Arg Ala Val Lys | |
| Asn Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Ser Leu Glu His | |
| Ala Gln Glu Gln Gln; | |
| Met Ala Asn Cys Ser Ile Met Ile Asp | SEQ ID NO:38 |
| Glu Ile Ile His His Leu Lys Arg Pro | |
| Pro Asn Pro Leu Leu Asp Pro Asn Asn | |
| Leu Asn Ser Glu Asp Met Asp Ile Leu | |
| Met Glu Arg Asn Leu Arg Thr Pro Asn | |
| Leu Leu Ala Phe Val Arg Ala Val Lys | |
| His Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:39 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Val Pro Cys | |
| Leu Pro Ser Ala Thr Ala Ala Pro Ser | |
| Arg His Pro Ile Thr Ile Lys Ala Gly | |
| Asp Trp Gln Glu Phe Arg Glu Lys Leu | |
| Thr Phe Tyr Leu Val Ser Leu Glu His | |
| Ala Gln Glu Gln Gln; | |
| Met Ala Asn Cys Ser Ile Met Ile Asp | SEQ ID NO:40 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Asp Arg Asn Leu Arg Leu Ser Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:41 |
| Glu Ala Ile His His Leu Lys Arg Pro | |
| Pro Ala Pro Ser Leu Asp Pro Asn Asn | |
| Leu Asn Asp Glu Asp Met Ser Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:42 |
| Glu Ile Ile His His Leu Lys Arg Pro | |
| Pro Ala Pro Leu Leu Asp Pro Asn Asn | |
| Leu Asn Asp Glu Asp Met Ser Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:43 |
| Glu Ile Ile His His Leu Lys Arg Pro | |
| Pro Ala Pro Leu Leu Asp Pro Asn Asn | |
| Leu Asn Ala Glu Asp Val Asp Ile Leu | |
| Met Asp Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:44 |
| Glu Ile Ile His His Leu Lys Arg Pro | |
| Pro Ala Pro Leu Leu Asp Pro Asn Asn | |
| Leu Asn Asp Glu Asp Val Ser Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Asn Cys Ser Ile Met Ile Asp | SEQ ID NO:45 |
| Glu Ile Ile His His Leu Lys Arg Pro | |
| Pro Ala Pro Leu Leu Asp Pro Asn Asn | |
| Leu Asn Asp Glu Asp Met Ser Ile Leu | |
| Met Glu Arg Asn Leu Arg Leu Pro Asn | |
| Leu Glu Ser Phe Val Arg Ala Val Lys | |
| Asn Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln; | |
| Met Ala Tyr Pro Glu Thr Asp Tyr Lys | SEQ ID NO:46 |
| Asp Asp Asp Asp Lys Asn Cys Ser Ile | |
| Met Ile Asp Glu Ile Ile His His Leu | |
| Lys Arg Pro Pro Ala Pro Leu Leu Asp | |
| Pro Asn Asn Leu Asn Ala Glu Asp Val | |
| Asp Ile Leu Met Glu Arg Asn Leu Arg | |
| Leu Pro Asn Leu Glu Ser Phe Val Arg | |
| Ala Val Lys Asn Leu Glu Asn Ala Ser | |
| Gly Ile Glu Ala Ile Leu Arg Asn Leu | |
| Gln Pro Cys Leu Pro Ser Ala Thr Ala | |
| Ala Pro Ser Arg His Pro Ile Ile Ile | |
| Lys Ala Gly Asp Trp Gln Glu Phe Arg | |
| Glu Lys Leu Thr Phe Tyr Leu Val Thr | |
| Leu Glu Gln Ala Gln Glu Gln Gln; | |
| Met Ala Tyr Pro Glu Thr Asp Tyr Lys | SEQ ID NO:47 |
| Asp Asp Asp Asp Lys Asn Cys Ser Ile | |
| Met Ile Asp Glu Ile Ile His His Leu | |
| Lys Arg Pro Pro Asn Pro Leu Leu Asp | |
| Pro Asn Asn Leu Asn Ser Glu Asp Met | |
| Asp Ile Leu Met Glu Arg Asn Leu Arg | |
| Thr Pro Asn Leu Leu Ala Phe Val Arg | |
| Ala Val Lys His Leu Glu Asn Ala Ser | |
| Gly Ile Glu Ala Ile Leu Arg Asn Leu | |
| Gln Pro Cys Leu Pro Ser Ala Thr Ala | |
| Ala Pro Ser Arg His Pro Ile Ile Ile | |
| Lys Ala Gly Asp Trp Gln Glu Phe Arg | |
| Glu Lys Leu Thr Phe Tyr Leu Val Thr | |
| Leu Glu Gln Ala Gln Glu Gln Gln; | |
| and | |
| Met Ala Asn Cys Ser Ile Met Ile Asp | SEQ ID NO:48 |
| Glu Leu Ile His His Leu Lys Ile Pro | |
| Pro Asn Pro Ser Leu Asp Ser Ala Asn | |
| Leu Asn Ser Glu Asp Val Ser Ile Leu | |
| Met Glu Arg Asn Leu Arg Thr Pro Asn | |
| Leu Leu Ala Phe Val Arg Ala Val Lys | |
| His Leu Glu Asn Ala Ser Gly Ile Glu | |
| Ala Ile Leu Arg Asn Leu Gln Pro Cys | |
| Leu Pro Ser Ala Thr Ala Ala Pro Ser | |
| Arg His Pro Ile Ile Ile Lys Ala Gly | |
| Asp Trp Gln Glu Phe Arg Glu Lys Leu | |
| Thr Phe Tyr Leu Val Thr Leu Glu Gln | |
| Ala Gln Glu Gln Gln. | |

6. The cultured stem cells of claim 1, 2, 3, 4 or 5 wherein is R₂ is R₁ or a hematopoietic growth factor selected from the group consisting of: GM-CSF, CSF-1, G-CSF, G-CSF Ser¹⁷, c-mpl ligand (MGDF or TPO), M-CSF, erythropoietin (EPO), IL-1, IL-2, IL-3, IL-4, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor, and stem cell factor (SCF);

7. The cultured stem cells of claim 6 wherein is R₂ is selected from the group consisting of G-CSF, G-CSF Ser¹⁷, flt3 ligand, and c-mpl ligand.

8. The cultured stem cells of claim 2 wherein said chimera protein is selected from group consisting of: SEQ ID NO:121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 165, 166, 167, and 168.

9. The cultured stem cells of claim 1, 2, 3, 4, 5, or 8 wherein said culture medium further comprises a hematopoietic growth factor selected from the group consisting of: GM-CSF, CSF-1, G-CSF, G-CSF Ser¹⁷, c-mpl ligand (MGDF or TPO), M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor and stem cell factor (SCF).

10. The cultured stem cells of claim 6 wherein said culture medium further comprises a hematopoietic growth factor selected from the group consisting of: GM-CSF, CSF-1, G-CSF, G-CSF Ser¹⁷, c-mpl ligand (MGDF or TPO), M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor and stem cell factor (SCF).

11. The cultured stem cells of claims 61 or 62 wherein said culture medium further comprises a hematopoietic growth factor selected from the group consisting of: GM-CSF, CSF-1, G-CSF, G-CSF Ser¹⁷, c-mpl ligand (MGDF or TPO), M-CSF, erythropoietin (EPO), IL-1, IL-4, IL-2, IL-5, IL-6, IL-7, IL-8, IL-9, IL-10, IL-11, IL-12, IL-13, IL-15, IL-16, LIF, flt3 ligand, human growth hormone, B-cell growth factor, B-cell differentiation factor, eosinophil differentiation factor and stem cell factor (SCF).
